(19) United States
(12) Patent Application Publication
(10) Pub. No.: US 2022/0333209 A1
(43) Pub. Date: Oct. 20, 2022

(54) CONDITIONAL TISSUE OF ORIGIN RETURN FOR LOCALIZATION ACCURACY

(71) Applicant: Grail, LLC, Menlo Park, CA (US)

(72) Inventors: Oliver Claude Venn, San Francisco, CA (US); Peter D. Freese, San Francisco, CA (US); Samuel S. Gross, Sunnyvale, CA (US); Robert Abe Paine Calef, Redwood City, CA (US); Arash Jamshidi, Redwood City, CA (US)

(21) Appl. No.: 17/714,062

(22) Filed: Apr. 5, 2022

Related U.S. Application Data
(60) Provisional application No. 63/171,355, filed on Apr. 6, 2021.

(51) Int. Cl. C12Q 1/6886; G16B 30/00; G16H 50/20

(52) U.S. Cl. CPC C12Q 1/6886; G16B 30/00; G16H 50/20; C12Q 2600/112; C12Q 2600/154

(57) ABSTRACT

Disclosed herein are systems and methods for localization of a disease state (e.g., tissue of origin of cancer) using nucleic acid samples. In an embodiment, a method comprises receiving a plurality of cancer signals of a sample, each cancer signal indicating a probability that the sample is associated with a different disease state of a plurality of disease states. The method determines a first cancer signal having a greatest probability among the plurality of cancer signals. In accordance with a determination that the first cancer signal satisfies a criterion, the method associates the sample with a first disease state. In accordance with a determination that the first cancer signal does not satisfy the criterion, the method determines a second cancer signal having a second greatest probability among the plurality of cancer signals, and associates the sample with the first disease state and a second disease state.

### CROSS REFERENCE TO RELATED APPLICATIONS

This application claims the benefit of priority to U.S. Provisional Application No. 63/171,355, filed on Apr. 6, 2021, which is incorporated herein by reference in its entirety for all purposes.

### BACKGROUND

### 1. Field of Art

This disclosure generally relates to conditional return of tissue of origin determinations for localization of disease states.

### 2. Description of the Related Art

A model can be trained to predict a tissue of origin of a suspected cancer. But due to biological ambiguity, there may be more than one plausible tissue of origin prediction. For example, biological samples with different tissues of origin of cancer may have similar features. It is difficult for a physician or another health care provider to parse ambiguous or complex cancer signals determine a diagnosis for an individual. Samples with low tumor shedding (e.g., early stage cancers) are also challenging to localize because there are fewer informative fragments.

### SUMMARY

Disclosed herein are methods for localization of a disease state (e.g., presence or absence of cancer, a cancer type, and/or a cancer tissue of origin (also referred to herein as “cancer signal origin”) using nucleic acid samples. The embodiments disclosed herein provide improvements to existing technology in the field of cancer diagnosis and early detection of cancer using non-invasive methods. In one aspect, the present disclosure provides a method for cancer diagnosis comprising: receiving a first plurality of cancer signals of a first sample of a first individual, wherein each one of the first plurality of cancer signals indicates a probability that the first sample is associated with a different disease state of a plurality of disease states; determining a first cancer signal having a greatest probability among the first plurality of cancer signals; responsive to determining that the first cancer signal satisfies a criterion associating the first sample with a disease state corresponding to the first cancer signal; providing, for presentation on a client device to determine a first diagnosis of the first individual, the disease state corresponding to the first cancer signal associated with the first sample; receiving a second plurality of cancer signals of a second sample of a second individual, wherein each one of the second plurality of cancer signals indicates a probability that the second sample is associated with a different disease state of the plurality of disease states; determining a second cancer signal having a greatest probability among the second plurality of cancer signals; responsive to determining that the second cancer signal does not satisfy the criterion , associating the second sample with a subset of the plurality of disease states corresponding to a subset of the second plurality of cancer signals including at least the second cancer signal; and providing, for presentation on the client device to determine a second diagnosis of the second individual, the subset of the plurality of disease states corresponding to the subset of the second plurality of cancer signals associated with the second sample.

In some embodiments, the method, system, or non-transitory computer readable medium of the present disclosure further comprises determining a third cancer signal having a second greatest probability among the second plurality of cancer signals, wherein the subset of the second plurality of cancer signals further includes the third cancer signal.

In some embodiments, the criterion is a probability threshold, wherein determining that the first cancer signal satisfies the criterion comprises determining that the greatest probability of the first cancer signal is greater than the probability threshold. In some embodiments, the probability threshold is at least 88%, 89%, 90%, 91%, or 92%.

In some embodiments, the method, system, or non-transitory computer readable medium of the present disclosure further comprises determining the criterion based on accuracy of cancer signal probabilities and false positives.

In some embodiments, the method, system, or non-transitory computer readable medium of the present disclosure further comprises determining the criterion based on residual risk of current cancer being associated with a sample.

In some embodiments, the method, system, or non-transitory computer readable medium of the present disclosure further comprises determining a subset of n cancer signals of the first plurality of cancer signals having the n greatest probabilities among the first plurality of cancer signals; and responsive to determining that at least a threshold number of the subset of the first plurality of cancer signals is associated with a category of disease states, associating the first sample with each disease state of the category of disease states.

In some embodiments, the category of disease states is human papillomavirus (HPV) cancer. In some embodiments, the category of disease states includes stomach cancer and intestinal cancer.

In some embodiments, the plurality of disease states includes a non-cancer state.

In some embodiments, the plurality of disease states includes one or more types of cancer selected from the group including anus cancer, breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, leukemia, kidney cancer, liver cancer, bile duct cancer, plasma cell neoplasm cancer, upper gastrointestinal tract cancer, vulvar cancer, and lung neuroendocrine tumors and other high-grade neuroendocrine tumors.

In some embodiments, the method, system, or non-transitory computer readable medium of the present disclosure further comprises providing, for presentation on the client device, a graphical comparison of each disease state corresponding to the subset of the plurality of disease states associated with the second sample. In some embodiments, the graphical comparison is a bar plot based on the probabilities of the second plurality of cancer signals.

In another aspect, the present disclosure provides a system comprising a computer processor and a memory, the memory storing computer program instructions that when executed by the computer processor cause the processor to perform steps comprising the steps of: receiving a first plurality of cancer signals of a first sample of a first individual, wherein each one of the first plurality of cancer signals indicates a probability that the first sample is associated with a different disease state of a plurality of disease states; determining a first cancer signal having a greatest probability among the first plurality of cancer signals; responsive to determining that the first cancer signal satisfies a criterion associating the first sample with a disease state corresponding to the first cancer signal; providing, for presentation on a client device to determine a first diagnosis of the first individual, the disease state corresponding to the first cancer signal associated with the first sample; receiving a second plurality of cancer signals of a second sample of a second individual, wherein each one of the second plurality of cancer signals indicates a probability that the second sample is associated with a different disease state of the plurality of disease states; determining a second cancer signal having a greatest probability among the second plurality of cancer signals; responsive to determining that the second cancer signal does not satisfy the criterion , associating the second sample with a subset of the plurality of disease states corresponding to a subset of the second plurality of cancer signals including at least the second cancer signal; and providing, for presentation on the client device to determine a second diagnosis of the second individual, the subset of the plurality of disease states corresponding to the subset of the second plurality of cancer signals associated with the second sample.

In another aspect, the present disclosure provides a non-transitory computer readable medium comprising instructions that, when executed by one or more processors, cause the one or more processors to perform steps comprising: receiving a first plurality of cancer signals of a first sample of a first individual, wherein each one of the first plurality of cancer signals indicates a probability that the first sample is associated with a different disease state of a plurality of disease states; determining a first cancer signal having a greatest probability among the first plurality of cancer signals; responsive to determining that the first cancer signal satisfies a criterion associating the first sample with a disease state corresponding to the first cancer signal; providing, for presentation on a client device to determine a first diagnosis of the first individual, the disease state corresponding to the first cancer signal associated with the first sample; receiving a second plurality of cancer signals of a second sample of a second individual, wherein each one of the second plurality of cancer signals indicates a probability that the second sample is associated with a different disease state of the plurality of disease states; determining a second cancer signal having a greatest probability among the second plurality of cancer signals; responsive to determining that the second cancer signal does not satisfy the criterion , associating the second sample with a subset of the plurality of disease states corresponding to a subset of the second plurality of cancer signals including at least the second cancer signal; and providing, for presentation on the client device to determine a second diagnosis of the second individual, the subset of the plurality of disease states corresponding to the subset of the second plurality of cancer signals associated with the second sample.

In another aspect, the present disclosure provides a method for cancer signal localization comprising: receiving a plurality of cancer signals of a sample, wherein each one of the plurality of cancer signals indicates a probability that the sample is associated with a different disease state of a plurality of disease states; determining a first cancer signal having a greatest probability among the plurality of cancer signals; in accordance with a determination that the first cancer signal satisfies a criterion, associating the sample with a first disease state corresponding to the first cancer signal; in accordance with a determination that the first cancer signal does not satisfy the criterion: determining a second cancer signal having a second greatest probability among the plurality of cancer signals, and associating the sample with the disease state corresponding to the first cancer signal and a second disease state corresponding to the second cancer signal.

In some embodiments, the method, system, or non-transitory computer readable medium of the present disclosure further comprises: in accordance with the determination that the first cancer signal satisfies the criterion, providing the first cancer signal as input to a machine learning model to determine a prediction of cancer in the sample; and in accordance with the determination that the first cancer signal does not satisfy the criterion, providing the first cancer signal and the second cancer signal as input to the machine learning model to determine the prediction of cancer in the sample.

In some embodiments, the method, system, or non-transitory computer readable medium of the present disclosure further comprises: in accordance with the determination that the first cancer signal satisfies the criterion, creating a first training set including the association of the sample with the first disease state corresponding to the first cancer signal to train a machine learning model for cancer signal localization; and in accordance with the determination that the first cancer signal does not satisfy the criterion, creating a second training set including the association of the sample with the first disease state corresponding to the first cancer signal and the second disease state corresponding to the second cancer signal to train the machine learning model.

In another aspect, the present disclosure provides a method for cancer signal localization comprising: receiving a plurality of cancer signals of a sample, wherein each one of the plurality of cancer signals indicates a probability that the sample is associated with a different disease state of a plurality of disease states; determining a first conditional probability that a first cancer signal of the plurality of cancer signals is a true positive given that remaining cancer signals of the plurality of cancer signals are incorrect; responsive to determining that the first conditional probability satisfies a criterion, associating the sample with at least a disease state corresponding to the first cancer signal; determining a subset of the plurality of cancer signals excluding the first cancer signal; determining a second conditional probability that a second cancer signal of the subset of the plurality of cancer signals is a true positive given that remaining cancer signals of the subset of the plurality of cancer signals are incorrect; and responsive to determining that the second conditional probability satisfies the criterion, associating the sample with at least a disease state corresponding to the second cancer signal.

In various embodiments, a system comprises a computer processor and a memory, the memory storing computer program instructions that when executed by the computer processor cause the processor to perform any of the methods described herein. In various embodiments, a non-transitory computer-readable medium stores one or more programs, the one or more programs including instructions which, when executed by an electronic device including a processor, cause the device to perform any of the methods described herein.

### BRIEF DESCRIPTION OF THE DRAWINGS

FIG. 1A is a flowchart of a method for cancer signal localization, according to various embodiments.

FIG. 1B is a flowchart of another method for cancer signal localization, according to various embodiments.

FIG. 2A illustrates a system for sequencing nucleic acid samples, according to various embodiments.

FIG. 2B is block diagram of an analytics system for cancer signal localization, according to various embodiments.

FIG. 3 is a flowchart describing a process of sequencing nucleic acids, according to various embodiments.

FIG. 4 illustrates experimental results of true positives and false positives during cancer signal localization, according to one embodiment.

FIG. 5 is a flowchart of a method for cancer signal localization based on conditional probability, according to various embodiments.

FIG. 6 illustrates experimental results of cancer signal localizations, according to an embodiment.

FIG. 7 illustrates experimental results of cancer signal localizations based on conditional return, according to an embodiment.

FIG. 8 illustrates experimental results of cancer signal localizations from occult cancer samples, according to an embodiment.

FIG. 9 is a plot illustrating subsampling of cancer samples, according to an embodiment.

FIGS. 10A and 10B illustrate detected cancer samples that are subsampled to match expected screening cancer signal strengths, according to an embodiment.

FIGS. 11A and 11B illustrate cancer signal strength, by cancer type, before and after subsampling, according to some embodiments.

FIG. 12 illustrates cancer signal strength, by cancer type and stage, before and after subsampling, according to some embodiments.

FIGS. 13A and 13B include bar graphs of the distribution of CSL call probabilities, such as the proportion of CSL signal captured by the first, second, third, and fourth CSL call, according to some embodiments.

FIGS. 14A and 14B include bar graphs of the distribution of CSL call probabilities, such as the proportion of CSL signal captured by the first, second, third, and fourth CSL calls, by actual cancer types, according to some embodiments.

FIGS. 15A, 15B, and 15C include bar graphs of median cancer scores, divided into false positives and true positives, according to some embodiments.

FIG. 16 illustrates cumulative probability scores, according to some embodiments.

FIGS. 17A and 17B illustrate conditional accuracy of cancer signal localizations according to some embodiments.

FIGS. 18A and 18B illustrate conditional accuracy of cancer signal localizations for solid and liquid sample types, according to some embodiments.

FIGS. 19A and 19B illustrate conditional accuracy of cancer signal localizations based on cancer stage, according to some embodiments.

FIGS. 20A and 20B illustrate cumulative accuracy of cancer signal localizations, according to some embodiments.

FIGS. 21A and 21B illustrate cancer signal localizations of false positives, according to some embodiments.

FIGS. 22A and 22B illustrate cancer signal localizations of false positives based on cancer type, according to some embodiments.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. It is also noted that the contents of all published materials (patent applications, patents, papers, conference proceedings, and the like) referenced herein are incorporated herein by reference in their entirety.

### I. DEFINITIONS

Unless defined otherwise, all technical and scientific terms used herein have the meaning commonly understood by a person skilled in the art to which this description belongs. As used herein, the following terms have the meanings ascribed to them below.

The term “individual” refers to a human individual. The term “healthy individual” refers to an individual presumed to not have a cancer or disease.

The term “subject” refers to an individual whose DNA is being analyzed. A subject may be a test subject whose DNA is be evaluated using whole genome sequencing or a targeted panel as described herein to evaluate whether the person has a disease state (e.g., cancer, type of cancer, or cancer tissue of origin). A subject may also be part of a control group known not to have cancer or another disease. A subject may also be part of a cancer or other disease group known to have cancer or another disease. Control and cancer/disease groups may be used to assist in designing or validating the targeted panel.

The term “reference sample” refers to a sample obtained from a subject with a known disease state.

The term “training sample” refers to a sample obtained from a known disease state that can be used to generate sequence reads. Training samples may be applied to probability models to generate features that can be utilized for disease state classification.

The term “test sample” refers to a sample that may have an unknown disease state.

The term “sequence read” refers to a nucleotide sequence read from a sample obtained from an individual. Sequence reads may be generated from nucleic acid fragments in the sample. A sequence read can be a collapsed sequence read generated from a plurality of sequence reads derived from a plurality of amplicons from a single original nucleic acid molecule. In some embodiments, the sequence read can be a deduplicated sequence read. Sequence reads can be obtained through various methods known in the art.

The term “disease state” refers to presence or non-presence of a disease, a type of disease, and/or a disease tissue of origin. For example, in one embodiment, the present disclosure provides methods, systems, and non-transitory computer readable medium for detecting cancer (i.e., presence or absence of cancer), a type of cancer, or a cancer tissue of origin.

The term “tissue of origin” or “TOO” refers to the organ, organ group, body region or cell type from which a disease state may arise or originate. For example, the identification of a tissue of origin or cancer cell type typically allows to identify appropriate next steps to further diagnose, stage, and decide on treatment.

The term “methylation” as used herein refers to a chemical process by which a methyl group is added to a DNA molecule. Two of DNA's four bases, cytosine (“C”) and adenine (“A”) can be methylated. For example, a hydrogen atom on the pyrimidine ring of a cytosine base can be converted to a methyl group, forming 5-methylcytosine. Methylation tends to occur at dinucleotides of cytosine and guanine referred to herein as “CpG sites.” In other instances, methylation may occur at a cytosine not part of a CpG site or at another nucleotide that is not cytosine; however, these are rarer occurrences. In this present disclosure, methylation is discussed in reference to CpG sites for the sake of clarity. However, the principles described herein are equally applicable for the detection of methylation in a non-CpG context, including non-cytosine methylation. For example, Adenine methylation has been observed in bacteria, plant and mammalian DNA, although it has received considerably less attention.

In such embodiments, the wet laboratory assay used to detect methylation may vary from those described herein as well known in the art. Further, the methylation state vectors may contain elements that are generally vectors of sites where methylation has or has not occurred (even if those sites are not CpG sites specifically). With that substitution, the remainder of the processes described herein are the same, and consequently the inventive concepts described herein are applicable to those other forms of methylation.

The term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

The term “cell free deoxyribonucleic nucleic acid,” “cell free DNA,” or “cfDNA” refers to deoxyribonucleic acid fragments that circulate in bodily fluids such blood, sweat, urine, or saliva and originate from one or more healthy cells and/or from one or more cancer cells.

The term “circulating tumor DNA” or “ctDNA” refers to deoxyribonucleic acid fragments that originate from tumor cells or other types of cancer cells, which may be released into an individual's bodily fluids such blood, sweat, urine, or saliva as result of biological processes such as apoptosis or necrosis of dying cells or actively released by viable tumor cells.

### II. OVERVIEW OF LOCALIZATION METHODS

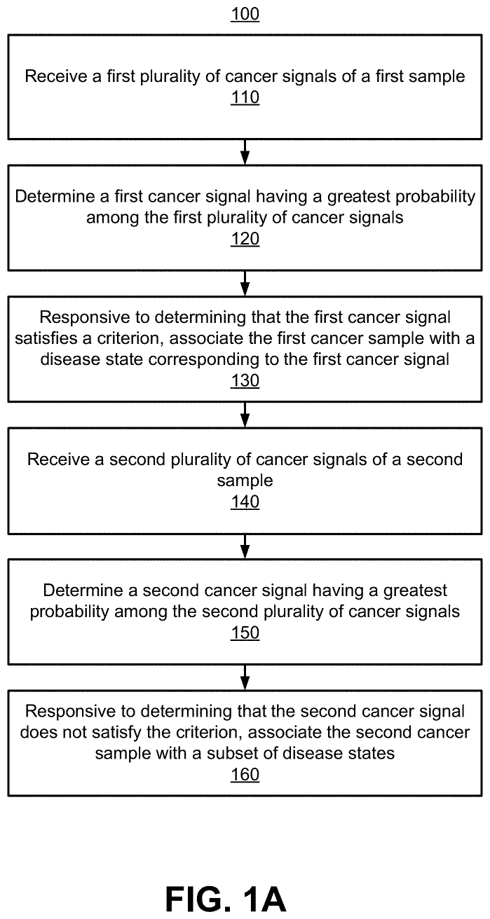

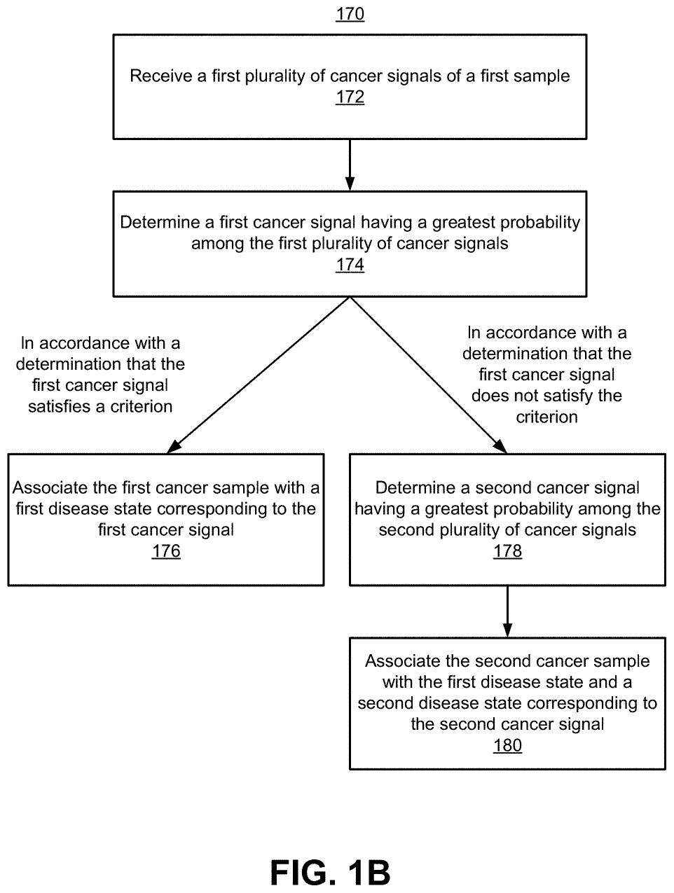

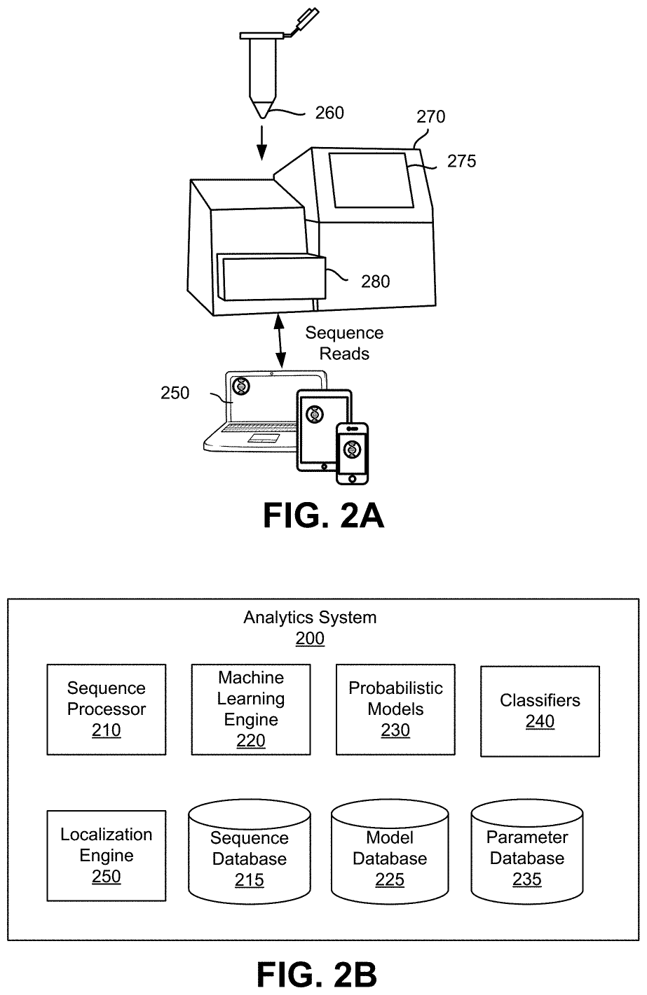

FIG. 1A is a flowchart of a method 100 for cancer signal localization, according to various embodiments. FIG. 2B is block diagram of an analytics system 200 for cancer signal localization, according to various embodiments. In the embodiment shown in FIG. 2B, the analytics system 200 includes a sequence processor 210, machine learning engine 220, probabilistic models 230, classifiers 240, and localization engine 250. In various embodiments, the analytics system 200 performs any of the methods described herein. The method 100 includes, but is not limited to, the following steps.

In step 110, the localization engine 250 receives a first set of cancer signals of a first sample. A cancer signal may also be referred to as a “probability score” or “cancer score.” Each cancer signal of the first set of cancer signals indicates a probability that the first sample is associated with a different disease state of a set of disease states. Each (probability of a) cancer signal may be on a scale from 0% to 100%, 0 to 100, 0 to 1. The cancer signals in the first set may sum to 100%, 100, or 1.

The cancer signals can be generated by one or more classifiers 240. In various embodiments, the classifier 240 generates the cancer signals by processing sequence reads of samples. The sequence processor 210 can generate the sequence reads of samples. In some embodiments, the signals are associated with disease states other than cancer. For example, the disease states can include medical or physiological conditions, genetic disorders, health-related metrics, and other types of diseases.

In various embodiments, a classifier 240 generates a set of 22 cancer signals, including cancer signals for 21 different cancer types and one non-cancer signal. The 21 different cancer types can include: Anus; Bladder and Urothelial Tract; Breast; Cervix; Colon and Rectum; Head and Neck; Kidney; Liver and Bile Duct; Lung; Neuroendocrine Cells of Lung or other Organs; Lymphoid Lineage; Melanocytic Lineage; Myeloid Lineage; Ovary; Pancreas and Gallbladder; Plasma Cell Lineage; Prostate; Bone and Soft Tissue; Thyroid Gland; Stomach and Esophagus; Uterus. In other embodiments, the classifier generates a set including a different number of cancer signals, or a set including different types of disease states than the list above.

In step 120, the localization engine 250 determines a first cancer signal having a greatest probability among the first set of cancer signals. In step 130, responsive to determining that the first cancer signal satisfies a criterion, the localization engine 240 associates the first sample with at least a disease state corresponding to the first cancer signal. For example, localization engine 250 can report a prediction that the first sample is associated with cancer having a tissue of origin indicated by the disease state. In some embodiments, the localization engine 250 only reports the disease state corresponding to the first cancer signal; that is, the localization engine 250 will not report predictions of disease states corresponding to the other cancer signals of the first set of cancer signals. Reporting only one disease state when the criterion is satisfied can help reduce complexity of output provided by the analytics system 200, which may assist a doctor's practice.

In various embodiments, the criterion is a 90% probability threshold of positive cancer scores. That is, the localization engine 250 determines whether the classifier 240 assigns 90% of the cancer signal tissue of origin score mass to the first cancer signal (corresponding to the disease state). In some embodiments where the set of cancer signals includes the 22 cancer types as previously described, the probability threshold does not account for the one non-cancer signal; that is, the localization engine 250 determines whether the classifier 240 assigns 90% of the cancer signal tissue of origin score mass among the 21 cancer signals to the first cancer signal. In other embodiments, the probability threshold does account for the one non-cancer signal in addition to the cancer signals indicating presence of cancer. In other embodiments, the criterion may be a different predetermined probability threshold, e.g., 88%, 89%, 91%, 92%, etc.

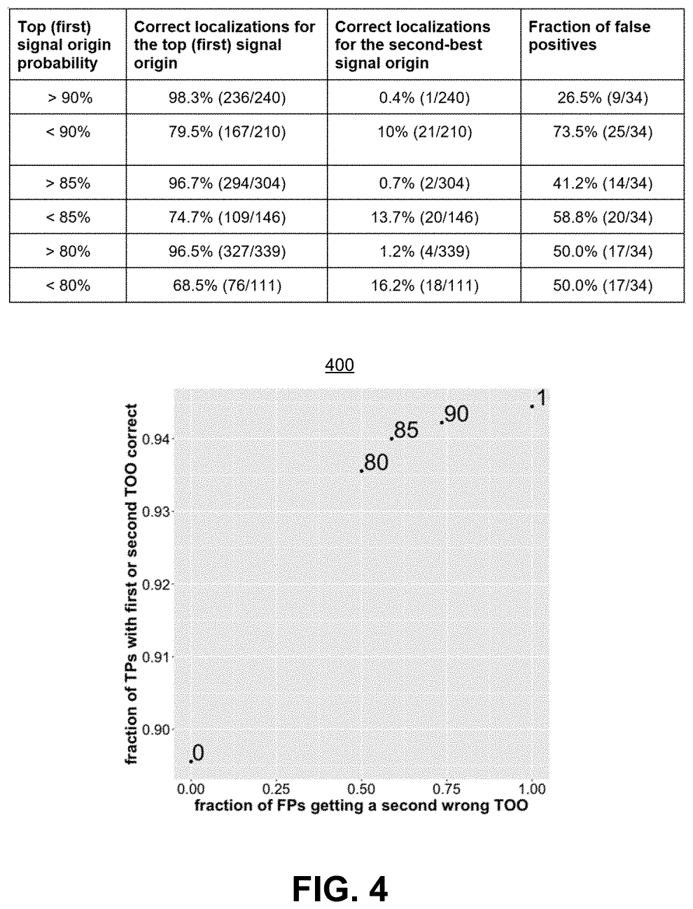

In various embodiments, the localization engine 250 determines the criterion based on accuracy of cancer signal probabilities and false positives. Selecting a probability threshold for the criterion that increases the fraction of true positives correctly detected can also increase the number of false positives, i.e., incorrectly predicting presence of cancer in a healthy sample that does not actually have presence of cancer. This trade-off is illustrated in the plot 400 illustrated in FIG. 4. At lower probability thresholds, the marginal benefit for true positive detection is high. At greater probability thresholds beyond 90%, the marginal benefit true positive detection is reduced, due to increased fraction of false positives. In an embodiment, the localization engine 250 determines the probability threshold by determining an inflection point of the curve on the plot 400 of true positive versus false positive detections. Based on the inflection point, the localization engine 250 determines that a probability threshold, 90% for example, is optimal because determining predictions of cancer using the probability threshold improves the accuracy of true positive detection while mitigating the risk of false positive detection. The probability threshold provides an improvement over conventional methods that do not consider the risk of false positives when making predictions of true positives. Conventional methods having a high rate of false positives result in a lower overall accuracy of predictions. Thus, the probability threshold is advantageous for the practical application of determining cancer predictions, particularly in non-invasive procedures, for example, using a blood sample instead of a tissue biopsy that would require surgery.

In step 140, the localization engine 250 receives a second set of cancer signals of a second sample. The first sample and second sample may be from two different patients or from the same patient. The samples can include any of cell free nucleic acid samples (e.g., cfDNA), solid tumor samples, and/or other types of biological samples. Each cancer signal of the second set of cancer signals indicates a probability that the second sample is associated with a different disease state of the set of disease states (e.g., the same set for the first set of cancer signals).

In step 150, the localization engine 250 determines a second cancer signal having a greatest probability among the second set of cancer signals. In step 160, responsive to determining that the second cancer signal does not satisfy the criterion, the localization engine 250 associates the second sample with a subset of the set of disease states corresponding to a subset of the second set of cancer signals. In some embodiments, the subset of the second set of cancer signals can include the cancer signals having the greatest two probabilities among the second set of cancer signals. In other embodiments, subset of the second set of cancer signals can include a different number of cancer signals, e.g., three, four, five, or more cancer signals.

In some embodiments, the localization engine 250 determines a subset of n cancer signals of the first set of cancer signals having the n greatest probabilities among the first set of cancer signals. Responsive to determining that at least a threshold number of the subset of the first set of cancer signals is associated with a category of disease states, the localization engine 250 associates the first sample with each disease state of the category of disease states. For example, the category of disease states is human papillomavirus (HPV) cancer. In a different example, the category of disease states includes stomach cancer and intestinal cancer. In other embodiments, the category of disease states can include one or more other types of cancer.

In some embodiments, the localization engine 250 can determine the criterion based on residual risk of current cancer being associated with a sample (risk of an individual being diagnosed with cancer). For example, the localization engine 250 determines to report an additional cancer signal based on a conditional probability of cancer given an incorrect tissue of origin prediction, where v is a ranked sorted vector of calibrated tissue of origin probabilities:

$$v = ( a 1 , a 2 , a 3 , … , a 21 ) \\ P ( false positive ) = 1 - v \\ P ( true positive with correct TOO ) = v * a 1 \\ P ( true positive with incorrect TOO ) = v * ( 1 - a 1 ) \\ P ( cancer \vert incorrect TOO ) = \frac{P ( true positive with incorrect TOO )}{P ( false positive ) + P ( true positive with incorrect TOO )}$$

The localization engine 250 can determine the probability that an individual has cancer after a cancer-positive test with no cancer detected at a first tissue of origin; cancer may be detected at a second or third tissue of origin.

The localization engine 250 can present disease state determinations (e.g., cancer tissue of origin localizations) to a user such as a doctor, physician, or clinician, among other types of health care providers. For example, the localization engine 250 provides the disease state corresponding to the first cancer signal associated with the first sample for presentation on a client device to a user. The localization engine 250 can provide a graphical comparison of each disease state corresponding to the subset of the set of disease states associated with the second sample. In various embodiments, the graphical comparison is a bar plot based on the probabilities of the second set of cancer signals. By presenting a visual depiction of the probabilities, a user can intuitively interpret the information output by the localization engine 250. For instance, the graphical comparison can suggest that the user place more weight on a tissue of origin having a greater probability of being a true positive tissue of origin of detected cancer.

FIG. 1B is a flowchart of another method 170 for cancer signal localization, according to various embodiments. The method 170 includes, but is not limited to, the following steps.

In step 172, the localization engine 250 receives a set of cancer signals of a sample. Each cancer signal of the set of cancer signals indicates a probability that the sample is associated with a different disease state of a set of disease states. In step 174, the localization engine 250 determines a first cancer signal having a greatest probability among the set of cancer signals.

In step 176, in accordance with a determination that the first cancer signal satisfies a criterion (such as any of the criterions described above), the localization engine 250 associates the sample with a first disease state corresponding to the first cancer signal.

In step 178, in accordance with a determination that the first cancer signal does not satisfy the criterion, the localization engine 250 determines a second cancer signal having a second greatest probability among the set of cancer signals; and in step 180, the localization engine 250 associates the sample with the disease state corresponding to the first cancer signal and a second disease state corresponding to the second cancer signal. In other words, the localization engine 250 associates the sample with the cancer signals having the greatest two probabilities among the second set of cancer signals.

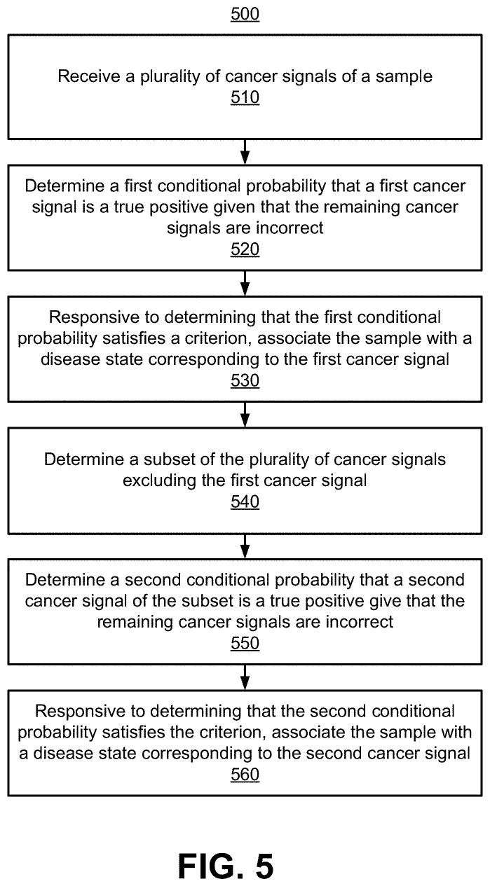

FIG. 5 is a flowchart of a method 500 for cancer signal localization based on conditional probability, according to various embodiments. Instead of using a predetermined probability threshold, the localization engine 250 can determine a threshold based on the conditional probability of an nth cancer signal being correct given that the previous n−1 cancer signals are incorrect. In this case, the localization engine 250 could continue to return cancer signals as long as P(nth cancer signal correct|previous n−1 cancer signals incorrect) satisfies a criterion such as exceeding a threshold probability. The method 500 includes, but is not limited to, the following steps.

In step 510, the localization engine 250 receives a set of cancer signals of a sample. Each of the cancer signals indicates a probability that the sample is associated with a different disease state of a set of disease states.

In step 520, the localization engine 250 determines a first conditional probability that a first cancer signal of the set of cancer signals is a true positive given that remaining cancer signals of the set of cancer signals are incorrect. In step 530, responsive to determining that the first conditional probability satisfies a criterion, the localization engine 250 associates the sample with at least a disease state corresponding to the first cancer signal.

In step 540, the localization engine determines a subset of the plurality of cancer signals excluding the first cancer signal. In step 550, the localization engine determines a second conditional probability that a second cancer signal of the subset of cancer signals is a true positive given that remaining cancer signals of the subset of cancer signals are incorrect. In step 560, responsive to determining that the second conditional probability satisfies the criterion, the localization engine 250 associates the sample with at least a disease state corresponding to the second cancer signal.

II.A. Assay Protocol

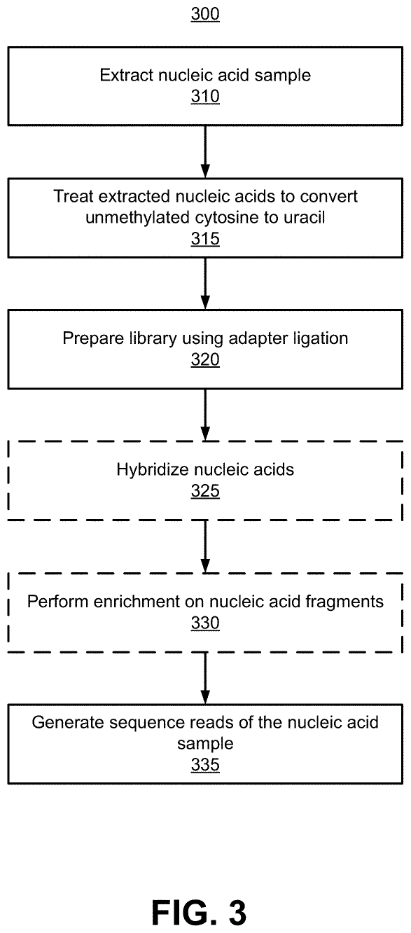

FIG. 3 is a flowchart describing a process 300 of sequencing nucleic acids, according to an embodiment. In some embodiments, the process 300 is performed to generate sequence reads used by the analytics system 200 to perform any of the methods for cancer signal localization described herein.

In step 310, a nucleic acid sample (e.g., DNA or RNA) is extracted from a subject. In the present disclosure, DNA and RNA can be used interchangeably unless otherwise indicated. That is, the embodiments described herein can be applicable to both DNA and RNA types of nucleic acid sequences. However, the examples described herein can focus on DNA for purposes of clarity and explanation. The sample can include nucleic acid molecules derived from any subset of the human genome, including the whole genome. The sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. If a subject has a disease state, such as cancer, cell free nucleic acids (e.g., cfDNA) in an extracted sample from the subject generally includes detectable level of the nucleic acids that can be used to assess a disease state.

In step 315, the extracted nucleic acids (e.g., including cfDNA fragments) are treated to convert unmethylated cytosines to uracils. In some embodiments, the method 300 uses a bisulfite treatment of the samples which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, Mass.).

In step 320, a sequencing library is prepared. In some embodiments, the preparation includes at least two steps. In a first step, a ssDNA adapter is added to the 3′-OH end of a bisulfite-converted ssDNA molecule using a ssDNA ligation reaction. In some embodiments, the ssDNA ligation reaction uses CircLigase II (Epicentre) to ligate the ssDNA adapter to the 3′-OH end of a bisulfite-converted ssDNA molecule, wherein the 5′-end of the adapter is phosphorylated and the bisulfite-converted ssDNA has been dephosphorylated (i.e., the 3′ end has a hydroxyl group). In another embodiment, the ssDNA ligation reaction uses Thermostable 5′ AppDNA/RNA ligase (available from New England BioLabs (Ipswich, MA)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA is synthesized in an extension reaction. For example, an extension primer, that hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter is added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 325, the nucleic acids (e.g., fragments) can be hybridized. Hybridization probes (also referred to herein as “probes”) may be used to target, and pull down, nucleic acid fragments informative for disease states. For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can cover overlapping portions of a target region.

In an optional step 330, the hybridized nucleic acid fragments are captured and can be enriched, e.g., amplified using PCR. In some embodiments, targeted DNA sequences can be enriched from the library. This is used, for example, where a targeted panel assay is being performed on the samples. For example, the target sequences can be enriched to obtain enriched sequences that can be subsequently sequenced. In general, any known method in the art can be used to isolate, and enrich for, probe-hybridized target nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

In step 335, sequence reads are generated from the nucleic acid sample, e.g., enriched sequences. Sequencing data can be acquired from the enriched DNA sequences by known means in the art. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

II.B. Exemplary Sequencer and Analytics System

FIG. 2A illustrates a system for sequencing nucleic acid samples, according to various embodiments. This illustrative diagram includes devices such as a sequencer 270 and an analytics system 200. The sequencer 270 and the analytics system 200 may work in tandem to perform one or more steps in the processes described herein.

In various embodiments, the sequencer 270 receives an enriched nucleic acid sample 260. As shown in FIG. 2A, the sequencer 270 can include a graphical user interface 275 that enables user interactions with particular tasks (e.g., initiate sequencing or terminate sequencing) as well as one more loading stations 280 for loading a sequencing cartridge including the enriched fragment samples and/or for loading necessary buffers for performing the sequencing assays. Therefore, once a user of the sequencer 270 has provided the necessary reagents and sequencing cartridge to the loading station 280 of the sequencer 270, the user can initiate sequencing by interacting with the graphical user interface 275 of the sequencer 270. Once initiated, the sequencer 270 performs the sequencing and outputs the sequence reads of the enriched fragments from the nucleic acid sample 260.

In some embodiments, the sequencer 270 is communicatively coupled with the analytics system 200. The analytics system 200 includes some number of computing devices used for processing the sequence reads for various applications such as assessing methylation status at one or more CpG sites, variant calling or quality control. The sequencer 270 may provide the sequence reads in a BAM file format to the analytics system 200. The analytics system 200 can be communicatively coupled to the sequencer 270 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 200 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. In one embodiment, the read pair R_1 and R_2 can be assembled into a fragment, and the fragment used for subsequent analysis and/or classification. An output file having SAM (sequence alignment map) format or BAM (binary) format may be generated and output for further analysis.

Referring now to FIG. 2B, the analytics system 200 implements one or more computing devices and/or one or more processors for use in analyzing DNA samples, sequence reads, or other information.

In some embodiments, the sequence processor 210 generates methylation state vectors for fragments from a sample. At each CpG site on a fragment, the sequence processor 210 generates a methylation state vector for each fragment specifying a location of the fragment in the reference genome, a number of CpG sites in the fragment, and the methylation state of each CpG site in the fragment whether methylated, unmethylated, or indeterminate. The sequence processor 210 may store methylation state vectors for fragments in the sequence database 215. Data in the sequence database 215 may be organized such that the methylation state vectors from a sample are associated to one another.

Further, multiple different models 230 may be stored in the model database 225 or retrieved for use with test samples. In one example, a model is a trained cancer classifier 240 for determining a cancer prediction for a test sample using a feature vector derived from anomalous fragments. The training and use of the cancer classifier is discussed elsewhere herein. The analytics system 200 may train the one or more models 230 and/or one or more classifiers 240 and store various trained parameters in the parameter database 235. The analytics system 200 stores the models 230 and/or classifiers 240 along with functions in the model database 225.

During inference, the machine learning engine 220 uses the one or more models 230 and/or classifiers 240 to return outputs. The machine learning engine accesses the models 230 and/or classifiers 240 in the model database 225 along with trained parameters from the parameter database 235. According to each model, the machine learning engine 220 receives an appropriate input for the model and calculates an output based on the received input, the parameters, and a function of each model relating the input and the output. In some use cases, the machine learning engine 220 further calculates metrics correlating to a confidence in the calculated outputs from the model. In other use cases, the machine learning engine 220 calculates other intermediary values for use in the model.

### III. MODEL BASED FEATURE ENGINEERING AND CLASSIFICATION

III.A. Model Based Feature Engineering

In accordance with one embodiment, the present disclosure is directed to model-based feature engineering for deriving features useful for classification of a disease state. As described elsewhere herein, the disease state can be the presence or absence of a disease, a type of disease, and/or a disease tissue or origin. For example, as described herein, the disease state can be the presence or absence of cancer, a type of cancer, and/or a cancer tissue of origin. The type of cancer and/or cancer tissue of origin can be selected from the group including breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, such as lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, among other types of cancer.

In a process, a first plurality of sequence reads are generated, as described elsewhere herein, from a first reference sample having a first disease state, and a second plurality of sequence reads are generated from a second reference sample having a second disease state. The first plurality of sequence reads and/or the second plurality of sequence reads can be more than 10,000, more than 50,000, more than 100,000, more than 200,000, more than 500,000, more than 1,000,000, more than 2,000,000, more than 5,000,000, or more than 10,000,000 sequence reads. As used herein a “reference sample” is a sample obtained from a subject with a known disease state. In some embodiments, one or more reference samples, having one or more known disease state, can be used to train one or more probabilistic models, that in turn can be used to derive features for classifying a disease state of an unknown test sample. The sample can be a genomic DNA (gDNA) sample or a cell free DNA (cfDNA) sample. The reference sample can be a blood, plasma, serum, urine, fecal, and saliva samples. Alternatively, the reference sample can be whole blood, a blood fraction, a tissue biopsy, pleural fluid, pericardial fluid, cerebral spinal fluid, and peritoneal fluid. In some embodiments, the first reference sample is obtained from a subject known to have cancer and the second reference sample is obtained from a healthy subject or a non-cancer subject. In some embodiments, the first reference sample is obtained from a subject known to have a first type of cancer (e.g., lung cancer) and the second reference sample is obtained from a subject known to have a second type of cancer (e.g., breast cancer). In still other embodiments, the first reference sample is obtained from a subject known to have a first disease tissue of origin (e.g., lung disease) and a second reference sample is obtained from a second disease state tissue of origin (e.g., a liver disease).

Continuing in the process, the machine learning engine 220 trains a first probabilistic model 230 and a second probabilistic model 230, from the first plurality of sequence reads and the second plurality of sequence reads, respectively, each probabilistic model associated with a different disease state of one or more possible disease states. As previously described, the disease state can be the presence or absence of cancer, a type of cancer, and/or a cancer tissue of origin. In various embodiments, training data is split into K subsets (folds) for K-fold cross-validation. Folds can be balanced for: cancer/non-cancer status, tissue of origin, cancer stage, age (e.g., grouped in 10-year buckets), gender, ethnicity, and smoking status, among other factors. Data from K−1 of the folds may be used as training data for the probabilistic models, and the held-out fold may be used as testing data.

The machine learning engine 220 trains the first and second probabilistic models 230, for the first and second disease states, respectively, by fitting each of the probabilistic models 230 to the first plurality and second plurality of sequence reads, respectively. For example, in one embodiment, the first probabilistic model is fitted using a first plurality of sequence reads derived from one or more samples from subjects known to have cancer and the second probabilistic model is fitted using the second plurality of sequence reads derived from one or more samples from healthy subjects or non-cancer subjects. In other embodiments, the first probabilistic model can be trained for a first type of cancer or a first tissue of origin and the second probabilistic model can be trained for a second type of cancer or a second tissue of origin. As one of skill in the art would appreciate, any number of disease state probabilistic models can be trained utilizing sequence reads derived from one or more sample taken from subjects with any one of a number of possible disease states. For example, in some embodiments, additional cancer-specific probabilistic models (i.e., for additional types of cancer and or tissues of origin models) can be trained for a third, fourth, fifth, sixth, seventh, eighth, ninth, tenth, etc. (e.g., up to twenty, thirty, or more) specific type of cancer and used to determine probabilities that sequence reads from a training set, or an unknown cancer type, are more likely derived from one cancer type (or cancer tissue of origin) than another cancer type (or cancer tissue of origin), as described elsewhere herein.

As used herein a “probabilistic model” is any mathematical model capable of assigning a probability to a sequence read based on methylation status at one or more sites on the read. During training, the machine learning engine 220 fits sequence reads derived from one or more samples from subjects having a known disease and can be used to determine sequence reads probabilities indicative of a disease state utilizing methylation information or methylation state vectors. In particular, in one embodiment, the machine learning engine 220 determines observed rates of methylation for each CpG site within a sequence read. The rate of methylation represents a fraction or percentage of base pairs that are methylated within a CpG site. The trained probabilistic model 230 can be parameterized by products of the rates of methylation. In general, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

III.B. Disease State Tissue of Origin Classification

In accordance with various embodiments, the machine learning engine 220 trains probabilistic models 230 each associated with a different disease state of a set of multiple disease states. As previously described, in various embodiments, the disease state can be the presence or absence of cancer, a type of cancer, and/or a cancer tissue of origin. Additionally, the disease state can be associated with another type of disease (not necessarily associated with cancer) or a healthy state (no presence of cancer or disease).

The machine learning engine 220 trains probabilistic models 230 using one or more sets of sequence reads, wherein each of the one or more sets of sequence reads are generated from a different disease state of the set of multiple disease states. The disease states can include any number of types of cancer or cancer tissues of origin selected from the group including breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, such as lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, among other types of cancer.

The machine learning engine 220 trains a probabilistic model 230, for each of the plurality of disease states, by fitting the probabilistic model 230 to the sequence reads deriving from each sample corresponding to each of the disease states. For example, in some embodiments, probabilistic models can be trained for specific types of cancer. In accordance with this embodiment, cancer-specific probabilistic models can be trained for a first, second, third, etc. specific type of cancer and used to assess a cancer type (e.g., of an unknown test sample). For example, a lung cancer-specific probabilistic model is fitted using a set of sequence reads deriving from one or more samples associated with lung cancer. As another example, a breast cancer-specific probabilistic model is fitted using a set of sequence reads deriving from one or more samples associated with breast cancer. In some embodiments, tissue specific probability models can be trained for a first, second, third, etc. tissue type and used to assess a disease state tissue of origin. For example, a first tissue of origin probabilistic model can be fitted using a set of sequence reads derived from a first tissue type (e.g., from a lung tissue sample, such as a lung biopsy) and a second tissue of origin probabilistic model can be fitted using a set of sequence reads derived from a second tissue type (e.g., from a liver tissue sample, such as a liver biopsy). Alternatively, in some embodiments, a cancer probabilistic model is fitted using a set of sequence reads derived from one or more samples from subjects known to have cancer and a non-cancer specific probabilistic model is fitted using a set of sequence reads derived from one or more samples from healthy subjects or non-cancer subjects. As one of skill in the art would appreciate, any number of disease state probabilistic models can be trained utilizing sequence reads derived from one or more sample taken from subjects with any one of a number of possible disease states. For example, in some embodiments, a plurality of sequence reads can be generated from a 3, 4, 5, 6, 7, 8, 9, 10, or more reference sample, each obtained from one or more subjects having a different disease state (e.g., different types of cancer), and used to train 3, 4, 5, 6, 7, 8, 9, 10, or more probabilistic models.

During training, the machine learning engine 220 can be trained on sequence reads indicative of a disease state utilizing methylation information or methylation state vectors. In particular, the machine learning engine 220 determines observed rates of methylation for each CpG site within a sequence read. The rate of methylation represents a fraction or percentage of base pairs that are methylated within a CpG site. The trained probabilistic model 230 can be parameterized by products of the rates of methylation. As previously described, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

In some embodiments, a Markov model, in which the probability of methylation at each CpG site is dependent on the methylation state at some number of preceding CpG sites in the sequence read, or nucleic acid molecule from which the sequence read is derived. See, e.g., U.S. patent application Ser. No. 16/352,602, entitled “Anomalous Fragment Detection and Classification,” and filed Mar. 13, 2019.

In some embodiments, the probabilistic model 230 is a “mixture model” fitted using a mixture of components from underlying models. For example, in some embodiments, the mixture components can be determined using multiple independent sites models, where methylation (e.g., rates of methylation) at each CpG site is assumed to be independent of methylation at other CpG sites. Utilizing an independent sites model, the probability assigned to a sequence read, or the nucleic acid molecule from which it derives, is the product of the methylation probability at each CpG site where the sequence read is methylated and one minus the methylation probability at each CpG site where the sequence read is unmethylated. In accordance with this embodiment, the machine learning engine 220 determines rates of methylation of each of the mixture components. The mixture model is parameterized by a sum of the mixture components each associated with a product of the rates of methylation. A probabilistic model Pr of n mixture components can be represented as:

$$Pr ( fragment \vert \{ {β}_{k i} , {f}_{k} \} ) = {\sum }_{k = 1}^{n} {f}_{k} {\prod }_{i} {β}_{k i}^{{m}_{i}} {( 1 - {β}_{k i} )}^{1 - {m}_{i}}$$

For an input fragment, mᵢ ϵ{0, 1} represents the fragment's observed methylation status at position i of a reference genome, with 0 indicating unmethylation and 1 indicating methylation. A fractional assignment to each mixture component k is fₖ, where fₖ≥0 and Σₖ₌₁ⁿ fₖ=1. The probability of methylation at position i in a CpG site of mixture component k is βₖᵢ. Thus, the probability of unmethylation is 1−βₖᵢ. The number of mixture components n can be 1, 2, 3, 4, 5, 6, 7, 8, 9, 10, etc.

In some embodiments, the machine learning engine 220 fits the probabilistic model 230 using maximum-likelihood estimation to identify a set of parameters {βₖᵢ, fₖ} that maximizes the log-likelihood of all fragments deriving from a disease state, subject to a regularization penalty applied to each methylation probability with regularization strength r. The maximized quantity for N total fragments can be represented as:

$${\sum }_{j}^{N} ln ( Pr ( {fragment}_{j} \vert \{ {β}_{k i} , {f}_{k} \} ) ) + r \cdot ln ( {β}_{k i} ( 1 - {β}_{k i} ) )$$

The analytics system 200 applies a probabilistic model 230 to calculate values for each sequence read of a second set of sequence reads. The values are calculated based at least on a probability that the sequence read (and corresponding fragment) originated from a sample associated with the disease state of the probabilistic model 230. The analytics system 200 can repeat this step for each of the different probabilistic models 230. In some embodiments, the analytics system 200 calculates the value using a log-likelihood ratio R with the fitted probabilistic models associated with certain disease states. Specifically, the log-likelihood ratio can be calculated using the probabilities Pr of observing a methylation pattern on the fragment for samples associated with the disease state and healthy samples:

$${R}_{d i s ease state} ( fragment ) ≡ ln ( \frac{Pr ( fragment \vert disease state )}{Pr ( fragment \vert healthy )} )$$

In other embodiments, the analytics system 200 can calculate the value using a different type of ratio or equation. The machine learning engine 220 can determine a fragment to be indicative of a disease state (e.g., cancer) based on whether at least one of the log-likelihood ratios considered against the various disease state is above a threshold value.

III.C. Classification

In various embodiments, the analytics system 200 generates a classifier 240 using the features. The classifier 240 is trained to predict, for an input sequence read from a test sample of a test subject, a tissue of origin associated with a disease state. The analytics system 200 can select a predetermined number (e.g., 1024) of top ranking features for each pair of disease states for training the classifier, e.g., based on the mutual information calculations or another calculated measure. The predetermined number may be treated as a hyperparameter selected based on performance in cross-validation. The analytics system 200 can also select features from regions of a reference genome determined to be more informative in distinguishing between the pair of disease states. In various embodiments, the analytics system 200 keeps the best performing tier for each region and for each cancer type pair (including non-cancer as a negative type).

In some embodiments, the analytics system 200 trains the classifier 240 by inputting sets of training samples with their feature vectors into the classifier 240 and adjusting classification parameters so that a function of the classifier 240 accurately relates the training feature vectors to their corresponding label. The analytics system 200 can group the training samples into sets of one or more training samples for iterative batch training of the classifier 240. After inputting all sets of training samples including their training feature vectors and adjusting the classification parameters, the classifier 240 can be sufficiently trained to label test samples according to their feature vector within some margin of error. The analytics system 200 can train the classifier 240 according to any one of a number of methods, for example, L1-regularized logistic regression or L2-regularized logistic regression (e.g., with a log-loss function), generalized linear model (GLM), random forest, multinomial logistic regression, multilayer perceptron, support vector machine, neural net, or any other suitable machine learning technique.

In various embodiments, the analytics system 200 trains a multinomial logistic regression classifier on the training data for a fold and generates predictions for the held-out data. For each of the K folds, the analytics system 200 trains one logistic regression for each combination of hyperparameters. An example hyperparameter is the L2 penalty, i.e., a form of regularization applied to the weights of the logistic regression. Another example hyperparameter is the topK, i.e., the number of high-ranking regions to keep for each tissue type pair (including non-cancer). For instance, where topK=16, the analytics system 200 keeps the top 16 regions per tissue type pair, as ranked by the mutual information procedure described herein. By following this procedure, the analytics system 200 can generate a prediction for each sample in the training set while ensuring that classifiers are not trained on the data for which predictions are generated.

In various embodiments, for each set of hyperparameters, the analytics system 200 evaluates performance on the cross-validated predictions of the full training set, and the analytics system 200 selects the set of hyperparameters with the best performance for retraining on the full training set. Performance may be determined based on a log-loss metric. The analytics system 200 can calculate log-loss by taking the negative logarithm of the prediction for the correct label for each sample, and then summing over samples. For instance, a perfect prediction of 1.0 for the correct label would result in a log-loss of 0 (lower is more accurate). To generate predictions for a new sample, the analytics system 200 can calculate feature values using the method described above, but restricted to features (region/positive class combinations) selected under the chosen topK value. The analytics system 200 can use the generated features to create a prediction using the trained logistic regression model.

In various embodiments, the analytics system 200 applies the classifier 240 to predict a tissue of origin of a test sample, where the tissue of origin is associated with one of the disease states. In some embodiments, the classifier 240 can return a prediction or likelihood for more than one disease state or tissue of origin. For example, the classifier 240 can return a prediction that a test sample has a 65% likelihood of having a breast cancer tissue of origin, a 25% likelihood of having a lung cancer tissue of origin, and a 10% likelihood of having a healthy tissue of origin. The analytics system 200 can further process the prediction values to generate a single disease state determination.

### IV. EXAMPLES

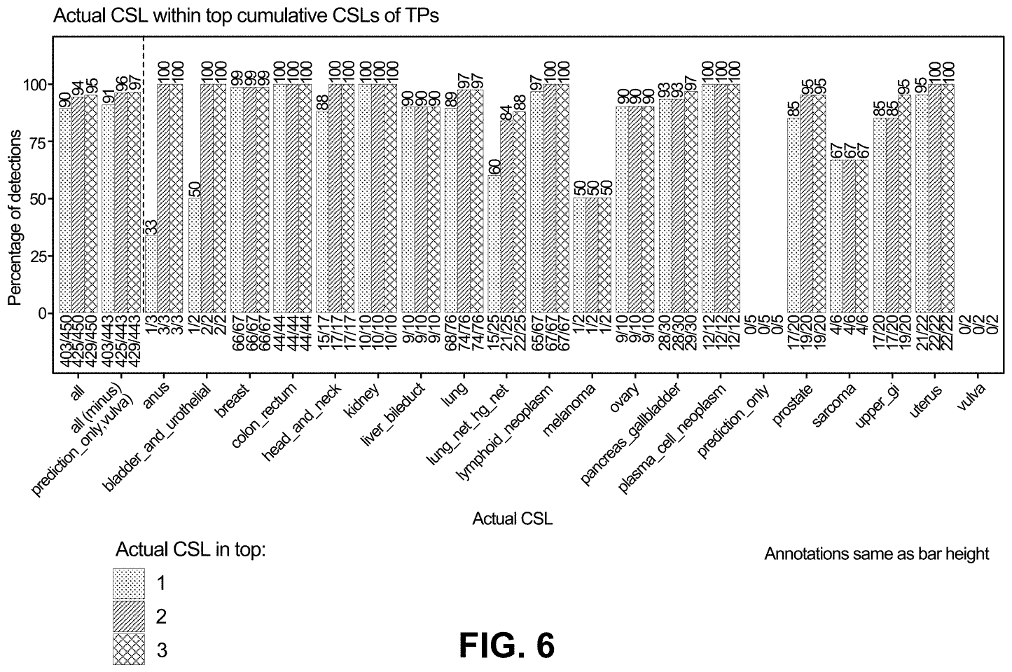

FIG. 6 illustrates experimental results of cancer signal localizations (“CSLs”), according to an embodiment. The experimental results indicate the percentage of cancer detections when the analytics system 200 reports one cancer signal (i.e., the cancer signal with the greatest probability score), two cancer signals (i.e., the cancer signals with the two greatest probability scores), and three cancer signals (i.e., the cancer signal with the three greatest probability scores). For many types of cancer included in the results, the percentage of detections increases when reporting two cancer signals instead of one cancer signal.

The experimental results are based on a set of 450 samples. These samples were chosen to reflect an expected distribution of cancer signal strength of occult cancers. Occult cancers are undiagnosed, pre-clinical cancers. Note that the subsample size for some cancer types such as anus and bladder & urothelial are small relative to the subsample size for other cancer types. FIG. 6 further demonstrates that if the first two CSLs were incorrect, the third CSL gives little detectable benefit in that of 5% of cases.

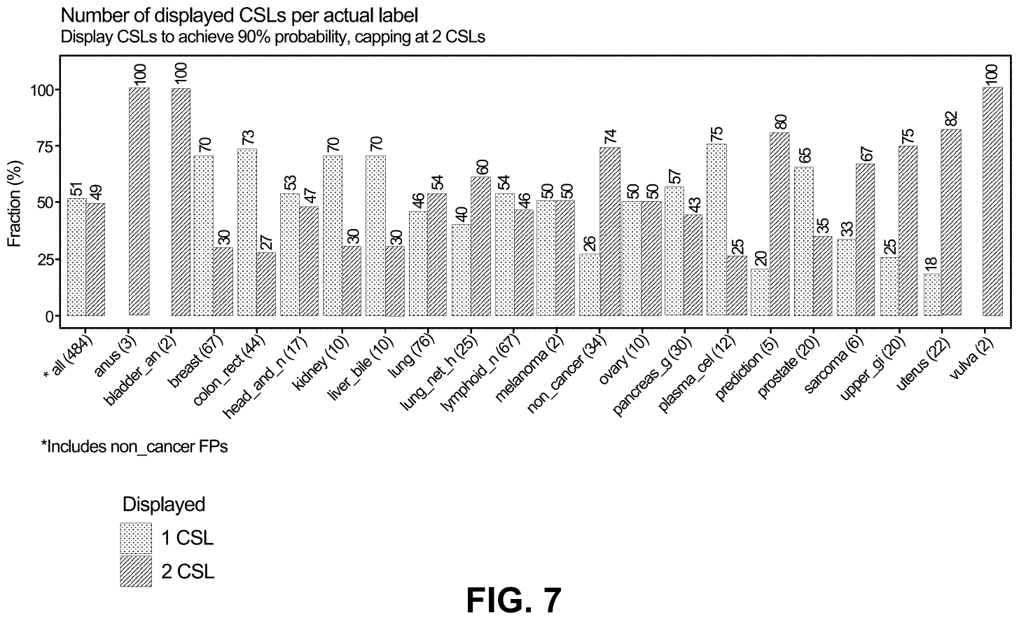

FIG. 7 illustrates experimental results of cancer signal localizations based on conditional return, according to an embodiment. Here, the analytics system 200 returns one cancer signal (the top scoring cancer signal) if the cancer signal has a probability score of 90% or greater of the positive cancer signal mass. Otherwise, the analytics system 200 returns at most the top two cancer signals, which are associated with the greatest two probability scores. The bar graph illustrates the fraction of samples under each type of cancer that had one and two cancer signals returned. For example, 70% of the breast cancer samples had one cancer signal returned, and 30% had two cancer signals returned. As another example, 50% of the ovary cancer samples had one cancer signal returned, and 50% had two cancer signals returned.

In summary, the experimental results indicated that the top CSL is correct approximately 90% of the cases, while the second CSL is correct half of the time when the top CSL is incorrect. The third CSL is wrong approximately 80% of the time when the top two are incorrect, and although better than chance, in some cases, it might not be useful towards facilitating doctors or other health care providers in making effective judgements, if reported. Therefore, in some embodiments, at most two localization attempts are provided, before other methods of diagnosis/analysis are embarked upon (e.g., full-body imaging). Notably, the results indicate that lymphoid and myeloid CSLs are localized very reliably, and that the majority of cancers are localized in the first two CSLs.

Reporting the top cancer signals using a determined probability threshold provides an improvement to existing cancer diagnosis processes because a health care provider is presented with a filtered subset of one or more cancer signals. The health care provider can determine a diagnosis more accurately and quickly by not having to parse through a larger set of signals that may include cancer signal localizations that are likely incorrect (e.g., false positives) or unreliable. As previously described, tumor shedding (e.g., early stage cancers) are challenging to localize because there are fewer informative fragments. Conventional methods for non-invasive cancer prediction thus have a difficult time handling false positives or unreliable cancer signals. Reducing this noise from the cancer signals reduces the complexity of the diagnosis process. Improved accuracy of cancer signal localizations also reduces unnecessary treatment for individuals having a false positive diagnosis of cancer.

In various embodiments, filtering cancer signals using a probability threshold also improves computer functionality because a method for cancer diagnosis uses the filtered cancer signals in subsequent processing steps. For example, the analytics system 200 uses the filtered (e.g., subset of) cancer signals as input to a machine learning model that outputs cancer predictions. As another example, the analytics system 200 uses the filtered cancer signals as training data to train the machine learning model to determine cancer predictions, e.g., the tissue of origin if presence of cancer is detected in a sample. In these examples, using the filtered cancer signals reduces the computational resources or processing time required by a computer implementing the machine learning model. The computer saves compute time by processing the top cancer signals (e.g., one or two signals of a subset determined by filtering using a probability threshold) instead of an unfiltered set of cancer signals. An unfiltered set of cancer signals may include ten or more cancer signals, as evident by the different cancer types shown in FIG. 7. Moreover, the unfiltered set of cancer signals would increase as additional cancer signals are identified over time. In various embodiments, the analytics system 200 processes cancer signals for many individuals. At large scale, the improvements to computer functionality are amplified due to the large size of data that the analytics system 200 must process to determine predictions of cancer. Determining cancer diagnosis more efficiently and quickly allows for earlier detection and treatment of cancer, which can be critical to an individual's health and prognosis. Achieving efficient and accurate predictions of cancer using non-invasive methods is further beneficial because these methods can make cancer diagnosis accessible to a greater population of individuals.

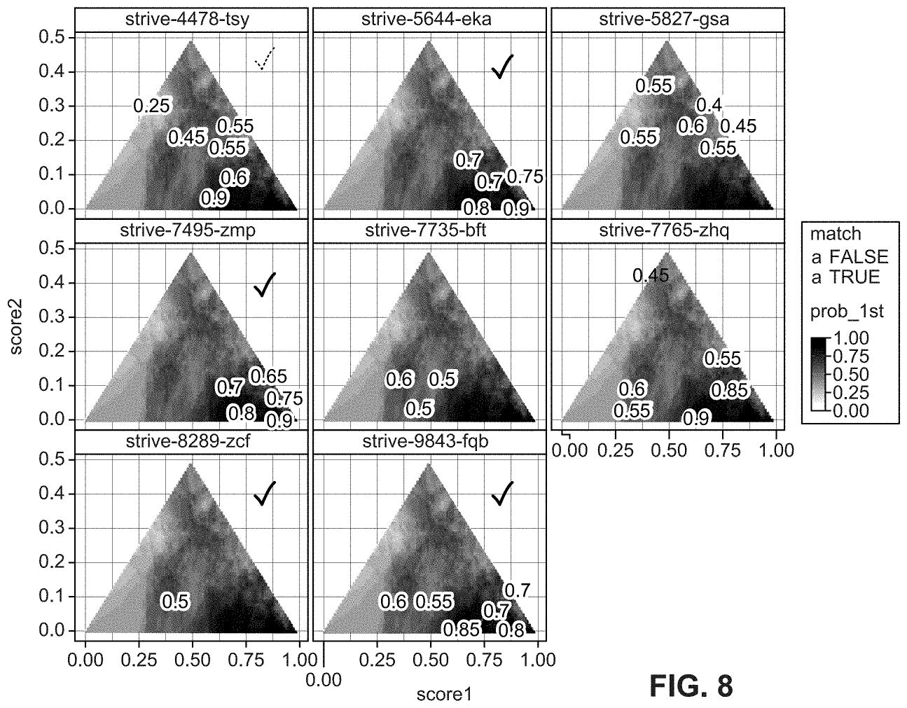

FIG. 8 illustrates experimental results of cancer signal localizations from occult cancer samples, according to an embodiment. The x-axis represents the first tissue of origin probability, and the y-axis represents the second tissue of origin probability. The occult cancer samples did not have diagnosed cancer during blood draw from individuals, but the individuals were later diagnosed with cancer. Thus, the cancer signal strengths from occult cancer samples are weaker relative to the signals from samples with cancers that have already been diagnosed. The cancer signal strengths from occult cancer samples also have greater uncertainty with respect to accuracy of tissue of origin localization.

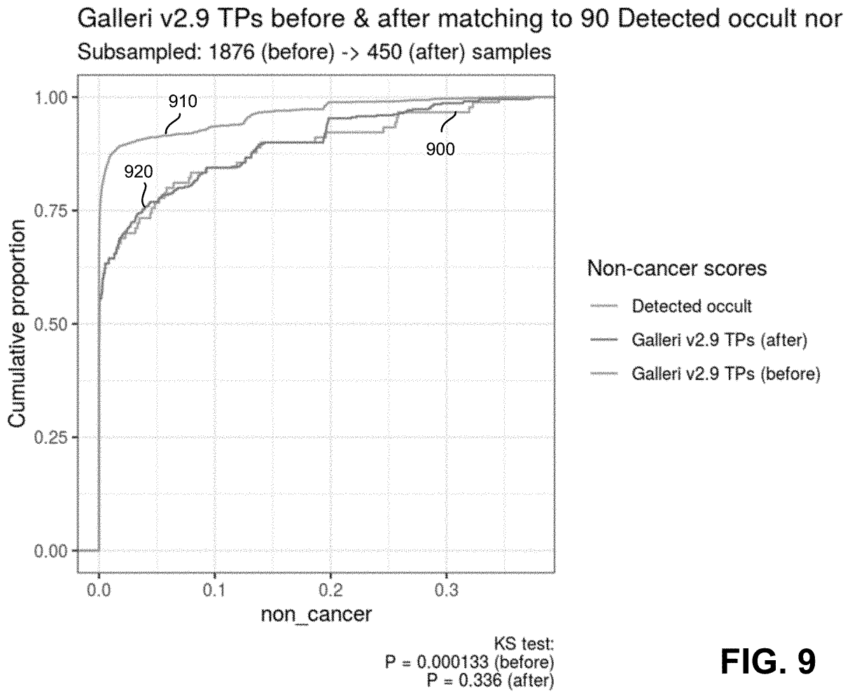

FIG. 9 is a plot illustrating subsampling of cancer samples, according to an embodiment. The proportion of true positive cancer detections for occult cancer samples 900 is lower relative to the proportion of true positive cancer detections for a set of diagnosed cancer samples 910. To more closely reflect the expected screening cancer signal strength of the occult cancer samples 900, the set of diagnosed cancer samples 910 (e.g., 1876 samples) was down-sampled to a subset of diagnosed cancer samples 920 (e.g., 450 samples). The subsampled true positives were selected based on matching to target occult non-cancer score within |Δnon_cancer score|<0.05, or |relative Δnon_cancer score|<0.1, or |Δlogit(non_cancer score)|<0.4, with empirically chosen thresholds that balance the tradeoff between how well occult distribution matched versus retaining sufficient number of samples for analysis.

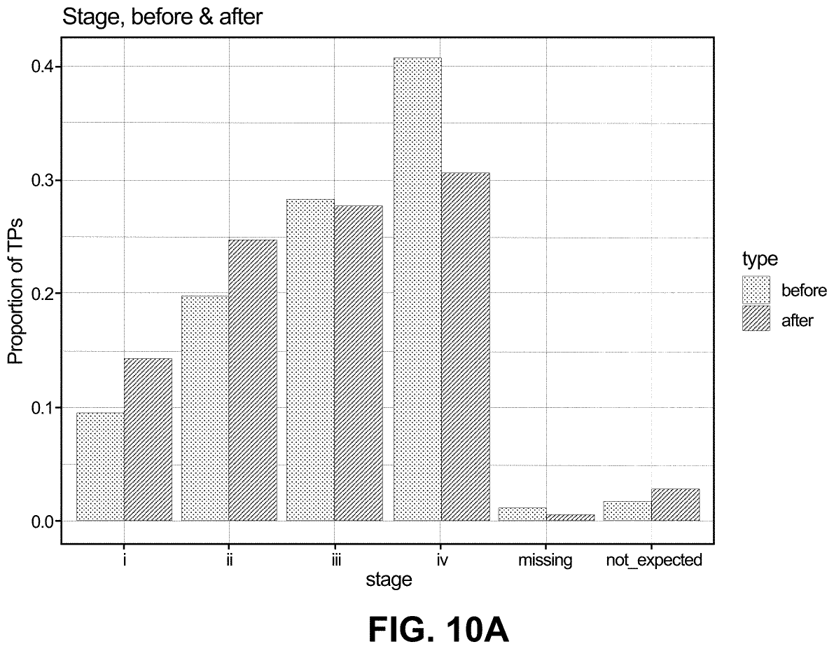

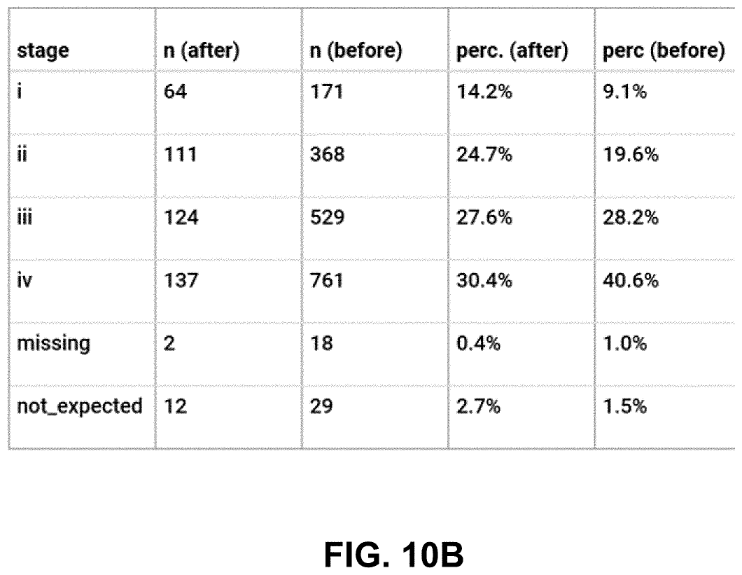

FIGS. 10A and 10B illustrate detected cancer samples (true positives) that are subsampled to match expected screening cancer signal strengths. The subsampling selects for fewer stage iv, and more stage i and ii cancers. Further, FIGS. 10A and 10B show cancer signal strength based on cancer stage, and that as the cancer stage progresses from stage i to stage iv, the proportion of true positives detected generally increases. However, in a comparison between two individuals, a sample from a first individual associated with stage i cancer could have a greater cancer signal strength than that of a sample from a second individual associated with stage iv cancer.

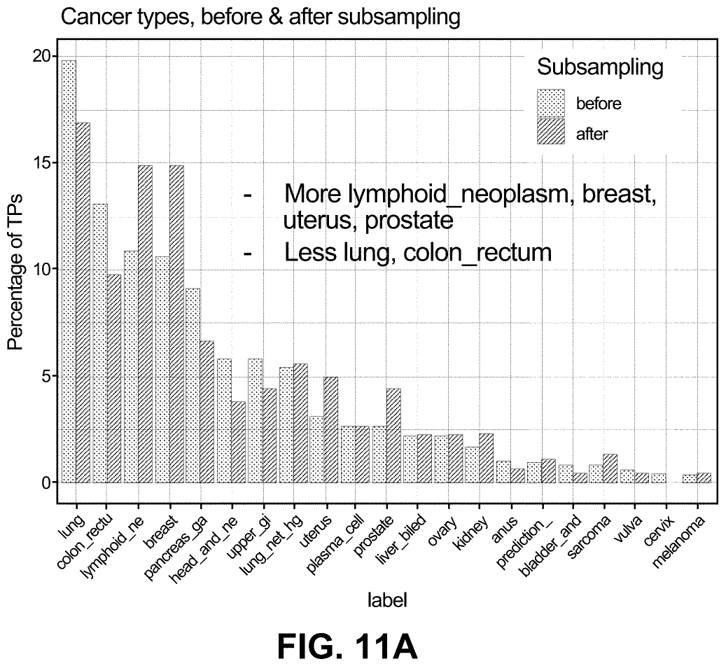

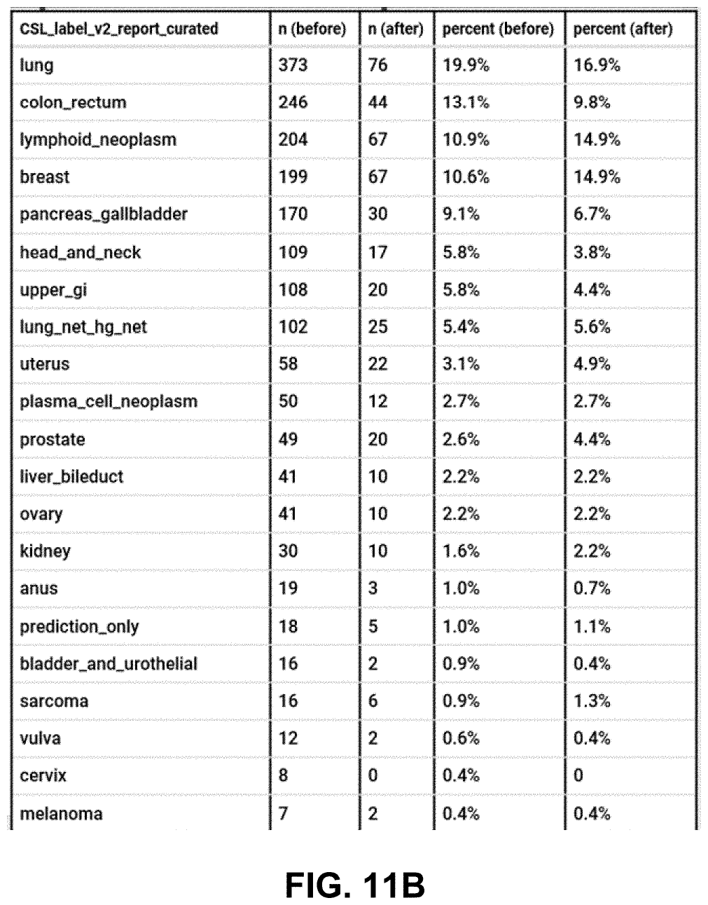

FIGS. 11A and 11B illustrate cancer signal strength, by cancer type, before and after subsampling, according to some embodiments. For some cancer types (e.g., lung, colon & rectum, and pancreas & gallbladder), the percentage of true positive detections decreased after subsampling. While for other cancer types (e.g., lymphoid neoplasms, breast, uterus, and prostate), the percentage of true positive detections increased after subsampling.

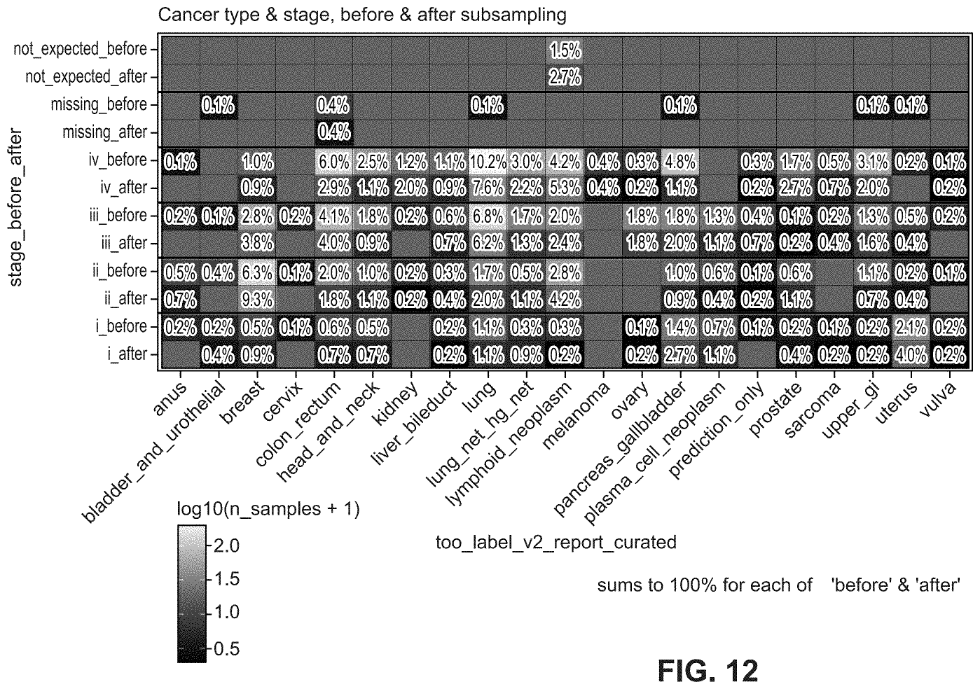

FIG. 12 illustrates cancer signal strength, by cancer type and stage, before and after subsampling, according to some embodiments. As shown in FIG. 12, the largest changes are a decrease in stage iv lung, pancreas_gallbladder, and colon_rectum, and an increase in stage ii breast and stage i uterus.

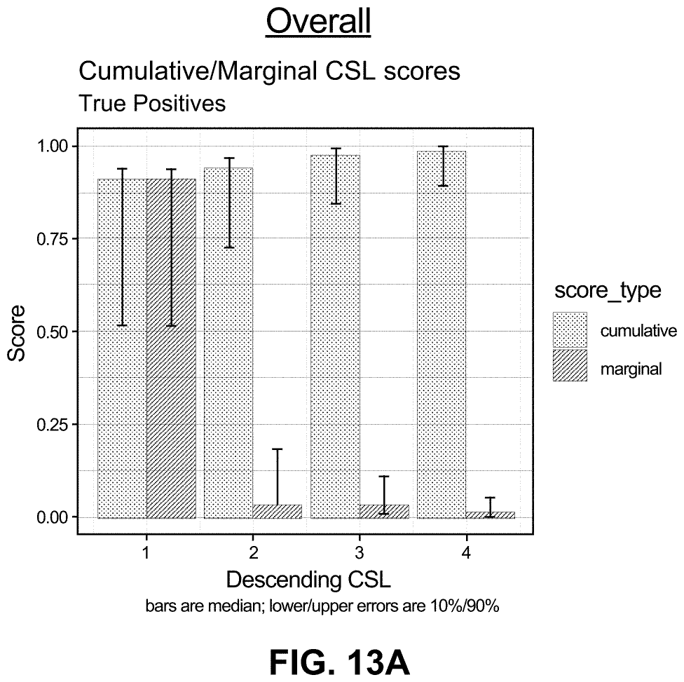

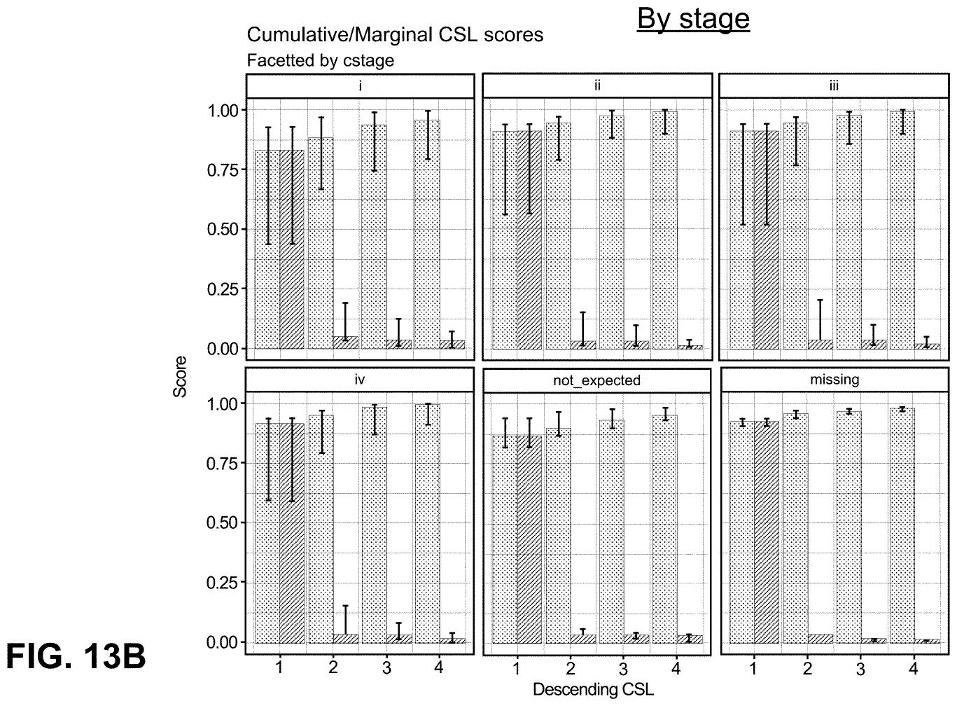

FIGS. 13A and 13B include bar graphs of the distribution of CSL call probabilities, such as the proportion of CSL signal captured by the first, second, third, and fourth CSL call, according to some embodiments. Specifically, FIG. 13A shows an overall graph of the distribution of cumulative and marginal cancer scores across the top four cancer signals. The cumulative bars reflect the sum of cancer scores for the top one, two, three, and/or four cancer signals. The bars are the median, with the lower and upper errors at 10% and 90%.

FIG. 13B shows graphs of the distribution of cumulative and marginal cancer scores across different cancer stages. The error bars in the bar graphs indicate the 10th and 90th percentile cancer scores. As shown in FIGS. 13A-13B, approximately 50-95% of the signal is captured in the top CSL, with a median at approximately 90%, and slightly less for early stages.

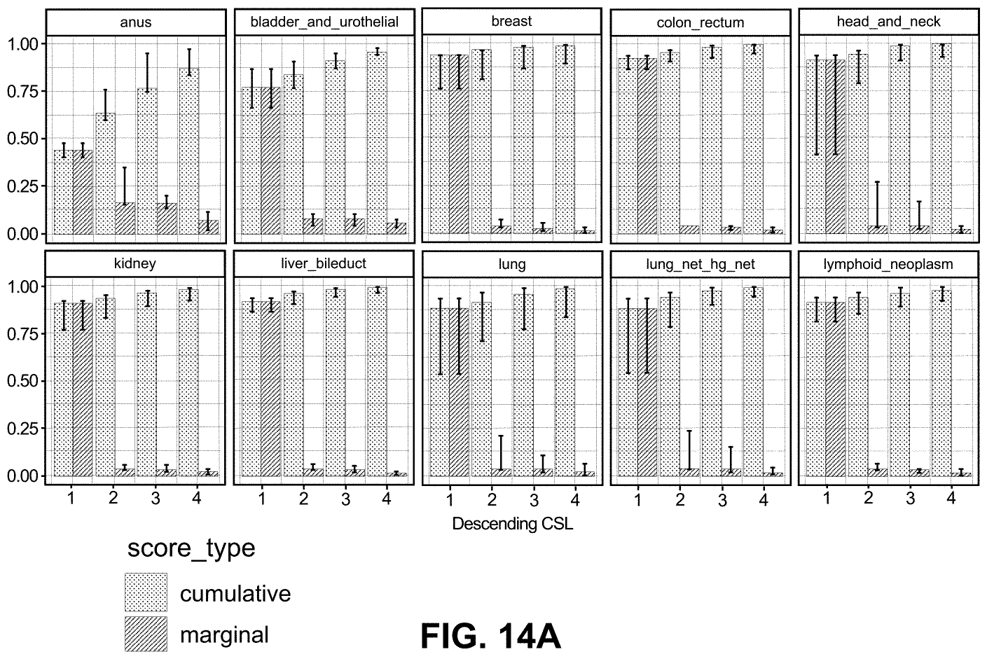

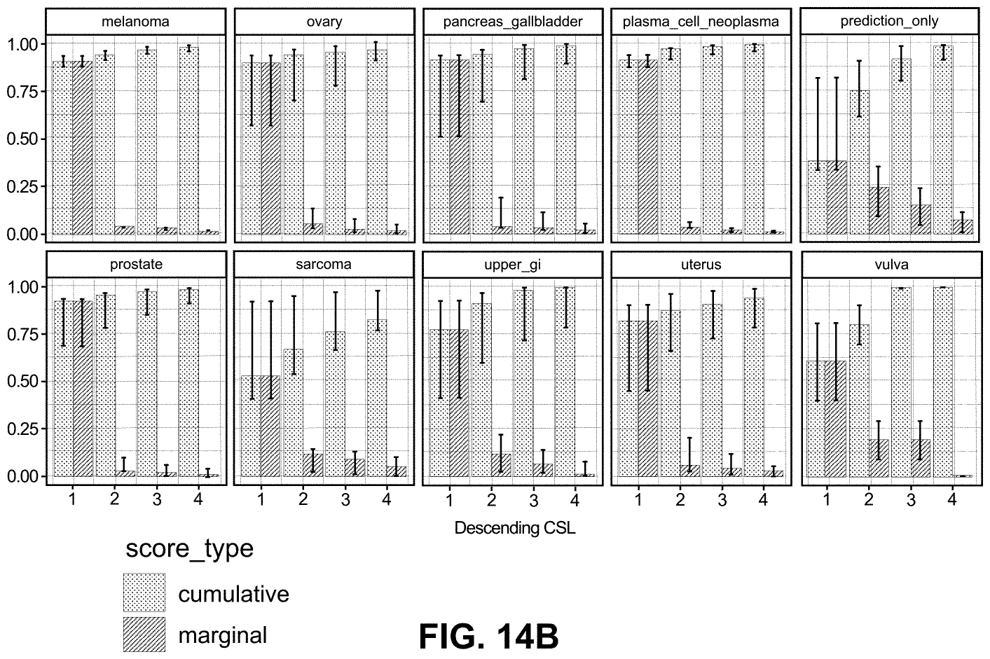

FIGS. 14A and 14B include bar graphs of the distribution of CSL call probabilities, such as the proportion of CSL signal captured by the first, second, third, and fourth CSL calls, by actual cancer types, according to some embodiments. As illustrated by the experimental results, samples of HPV-driven cancers such as anus and vulva have cancer scores that are lower in comparison to the cancer scores of other cancer types

In some embodiments, the localization engine 250 returns multiple cancer tissue of origins from a category (e.g., HPV-driven cancers) even if a top cancer score of an individual type of cancer within the category itself does not satisfy a criterion. For example, the top cancer signal of the anus samples has a cancer score 45% and the top cancer score of the vulva samples has a cancer score of 60%. Although neither cancer score satisfies a 90% probability threshold, the localization engine 250 can determine to return the anus and vulva cancer signals if the anus and vulva cancer signals are within a set of cancer signals having the greatest signal strength (e.g., the top three cancer signals). The localization engine 250 can condition the return of cancer signals based on other categories including multiple types of cancers (e.g., stomach cancer and intestinal cancer).

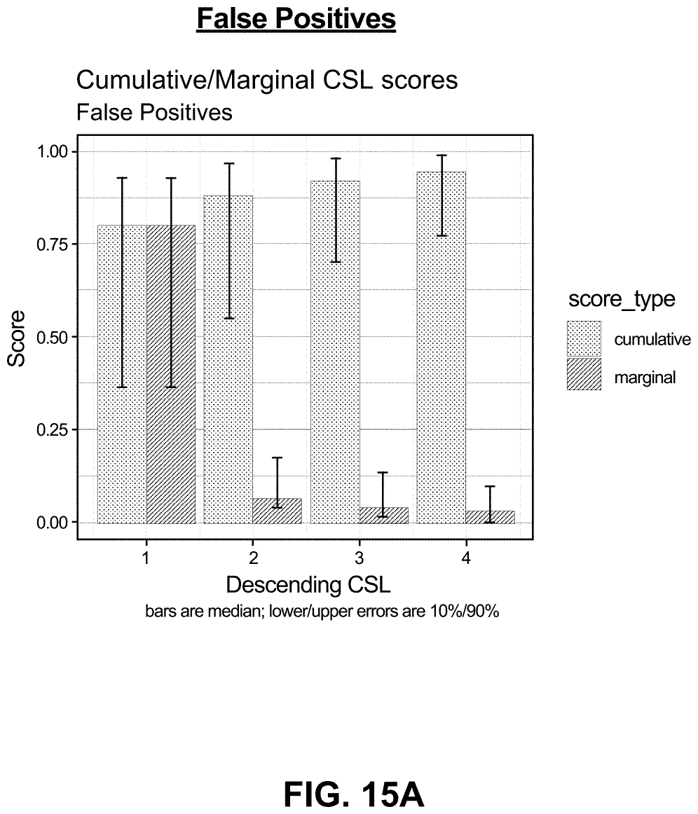

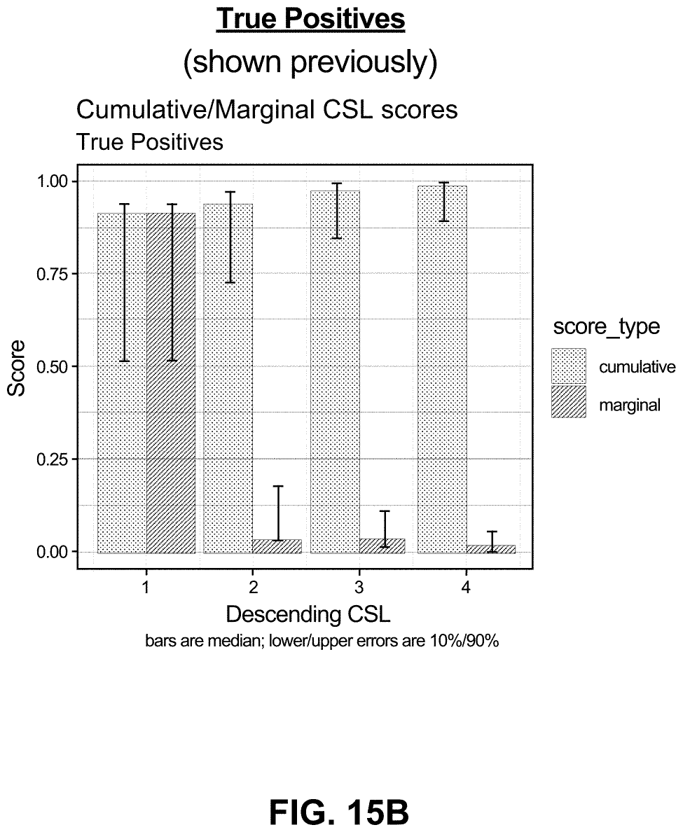

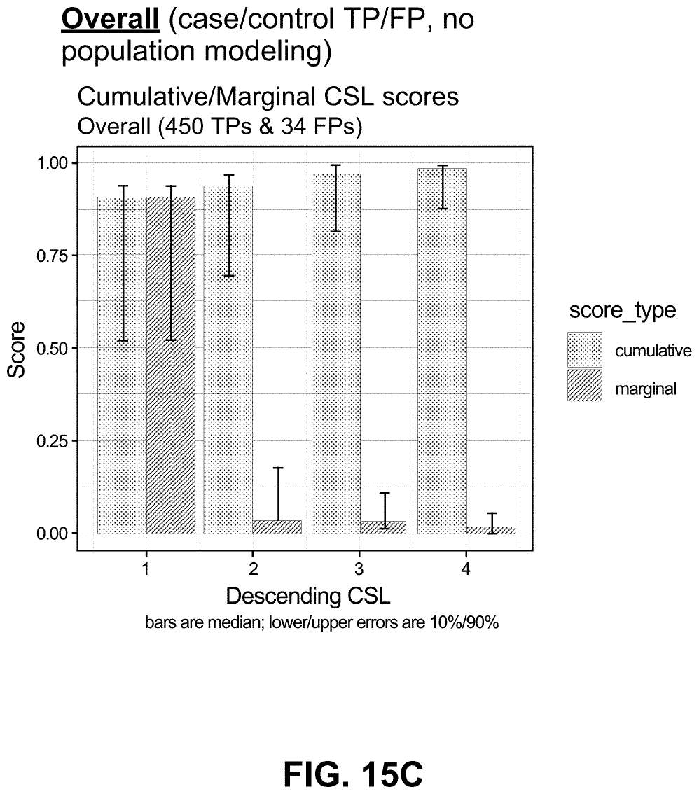

FIGS. 15A, 15B, and 15C include bar graphs of median cancer scores, divided into false positives and true positives, according to some embodiments. The magnitudes of cancer scores of false positives shown in FIG. 15A are lower than the magnitudes of cancer scores of true positives shown in FIG. 15B. Thus, the localization engine 250 more frequently returns two or more cancer signals for the false positives because the top cancer signal is less likely to meet a probability threshold (e.g., 90%).

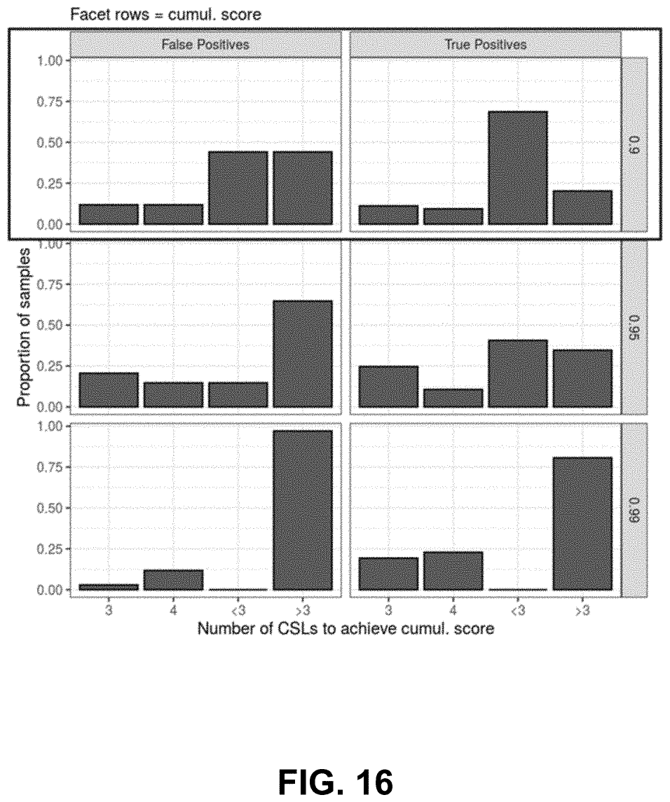

FIG. 16 illustrates cumulative probability scores, according to some embodiments. The plots in FIG. 16 show the number of cancer signals that would need to be returned by the localization engine 250 have their cumulative probability scores reach a threshold probability. For example, close to 75% of the true positive samples would require less than three cancer signals returned (i.e., one or two cancer signals returned) to accumulate a threshold probability of 90%. In contrast, less than 50% of the false positive samples would require less than three cancer signals returned to accumulate a threshold probability of 90%. These results are consistent with the results shown in FIGS. 15A-C because the cancer scores of false positives tend to be lower than the magnitudes of cancer scores of true positives.

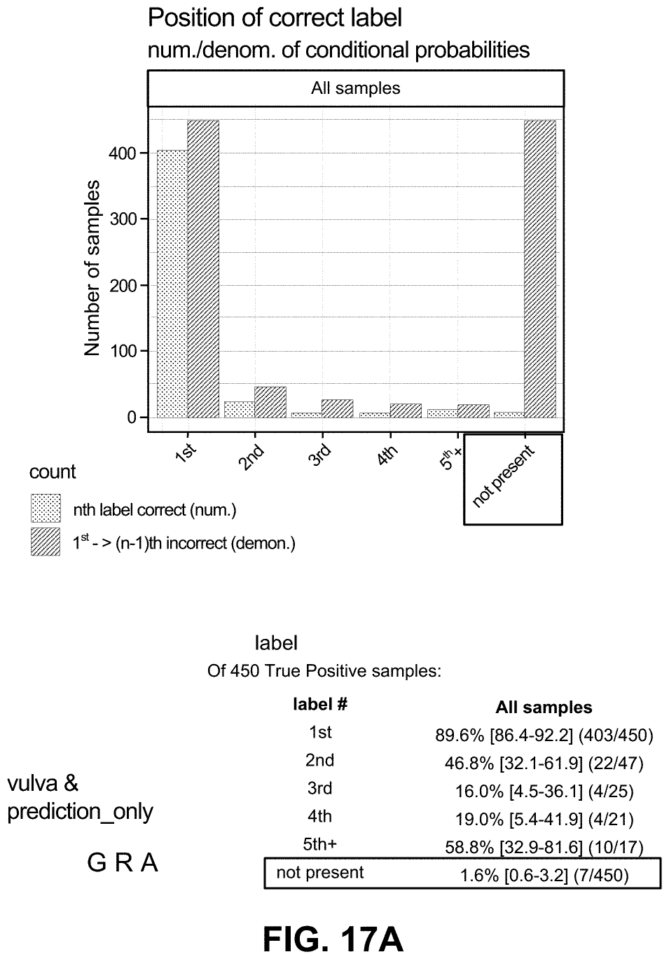

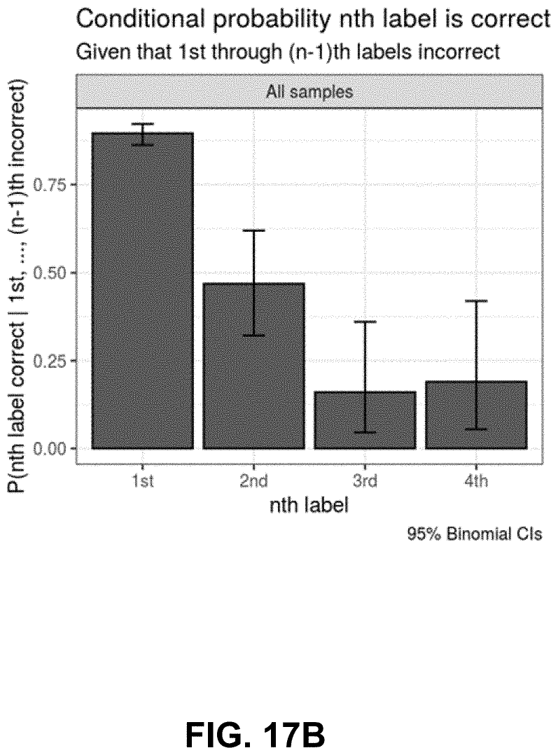

FIGS. 17A and 17B illustrate conditional accuracy of cancer signal localizations according to some embodiments. As shown in FIG. 17B, the top cancer signal (i.e., 1st label having the greatest probability score) is correct in approximately 90% of the samples. The second cancer signal (i.e., 2nd label) is correct in approximately 50% of the samples when the top cancer signal is incorrect. The third cancer signal (i.e., 3rd label) is correct in approximately 20% of the samples when the top two cancer signals are incorrect.

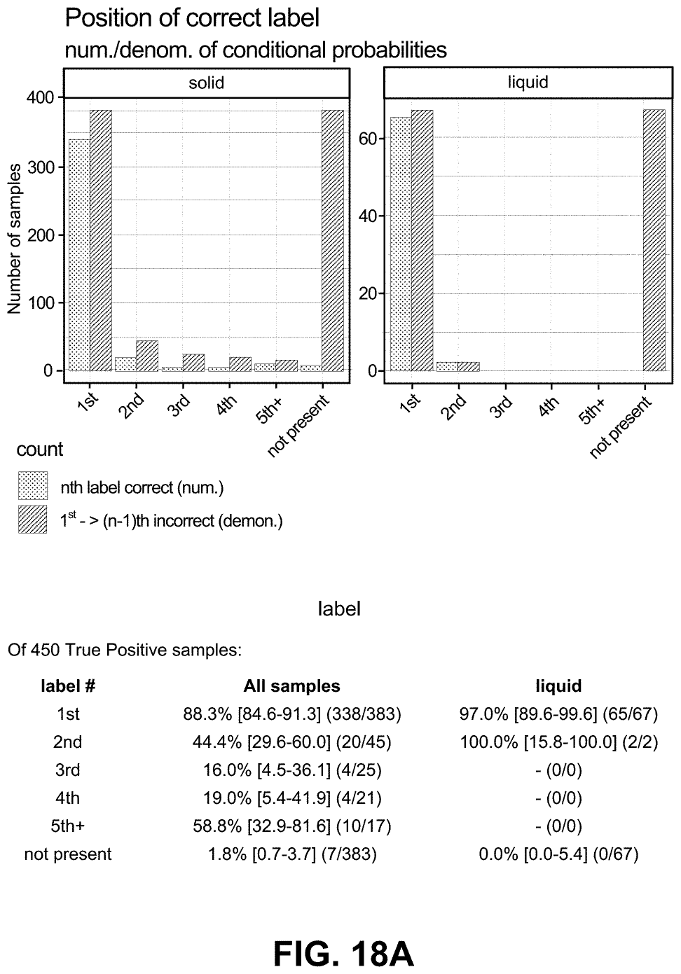

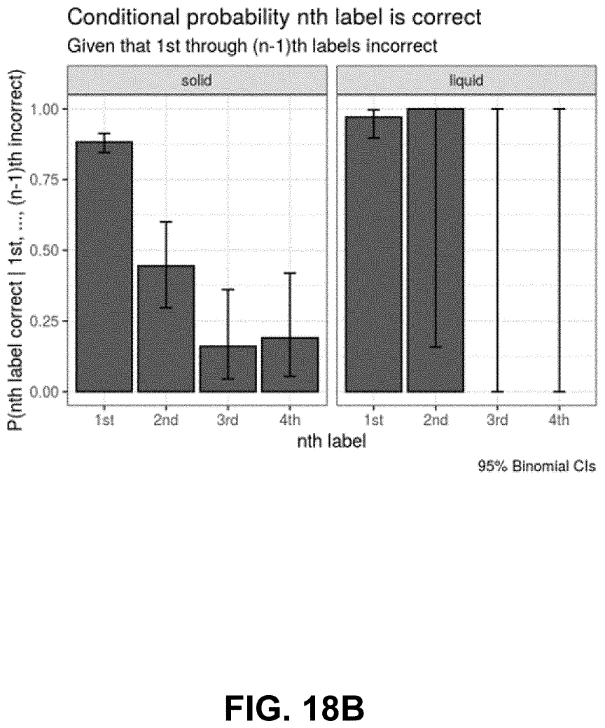

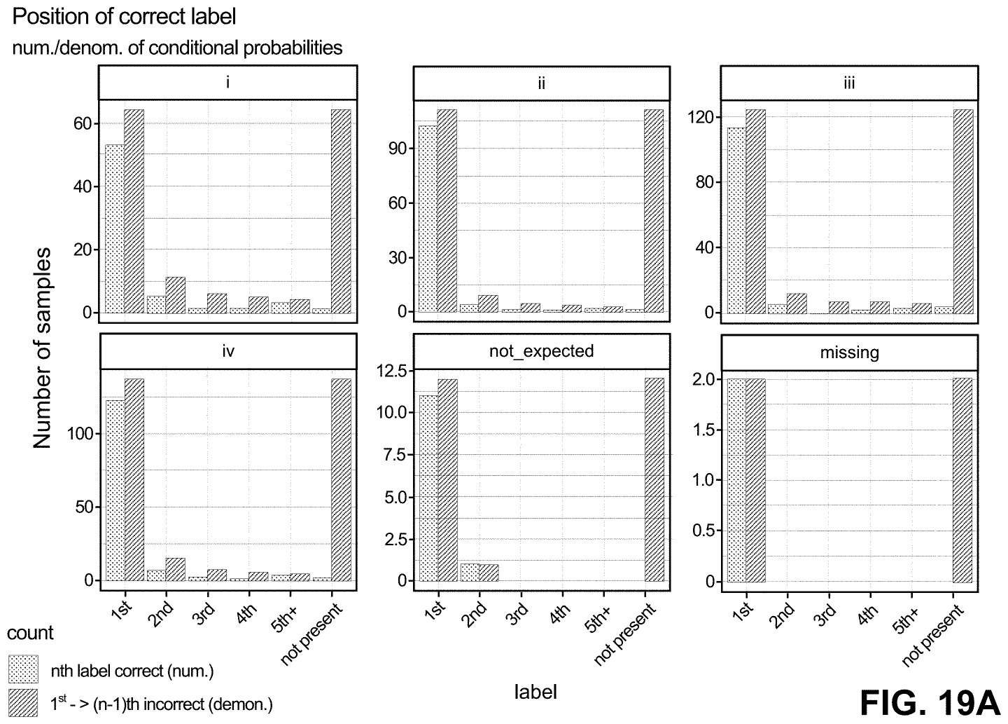

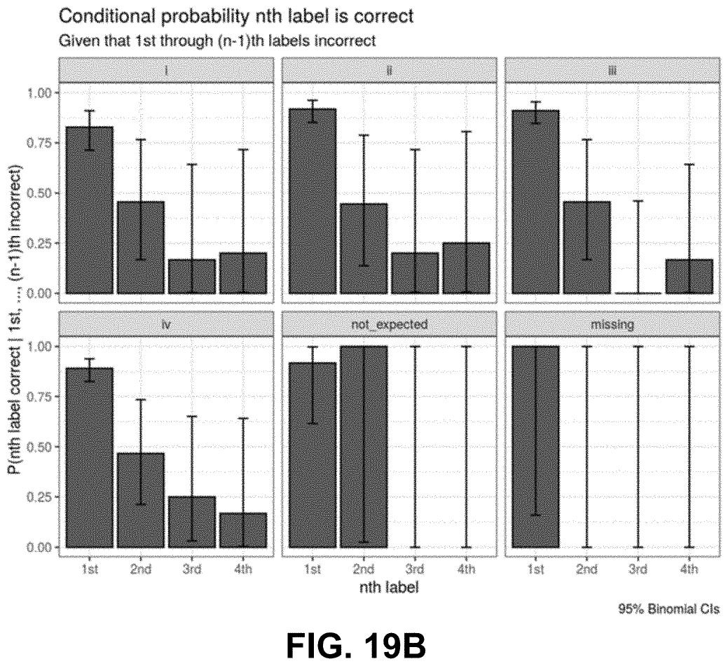

FIGS. 18A and 18B illustrate conditional accuracy of cancer signal localizations for solid and liquid sample types, according to some embodiments. FIGS. 19A and 19B illustrate conditional accuracy of cancer signal localizations based on cancer stage, according to some embodiments. The results in FIG. 18A show that the cancer signal localizations of liquid samples are more accurate than those of the solid samples. In comparison to the solid samples, for a greater number of the liquid samples, the localization engine 250 returned a top cancer signal (i.e., 1st label) that was a correct localization of cancer tissue of origin. In contrast, correct localization for the solid samples required more cancer signals (i.e., 2nd, 3rd, 4th, 5th+ labels) to be returned.

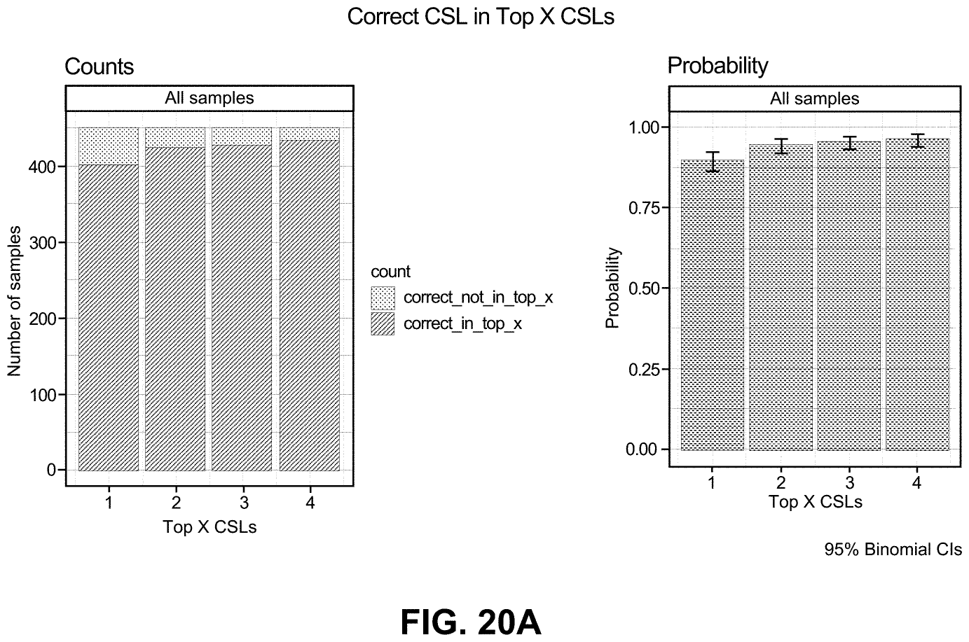

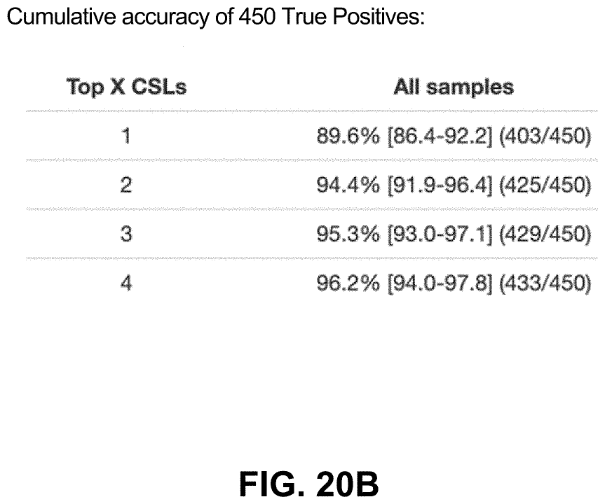

FIGS. 20A and 20B illustrate cumulative accuracy of cancer signal localizations, according to some embodiments. The top cancer signal is an accurate localization of tissue of origin in approximately 90% of the samples. The cumulative accuracy increases to approximately 94%, 95%, and 96% for the second, third, and fourth cancer signal localizations, respectively.

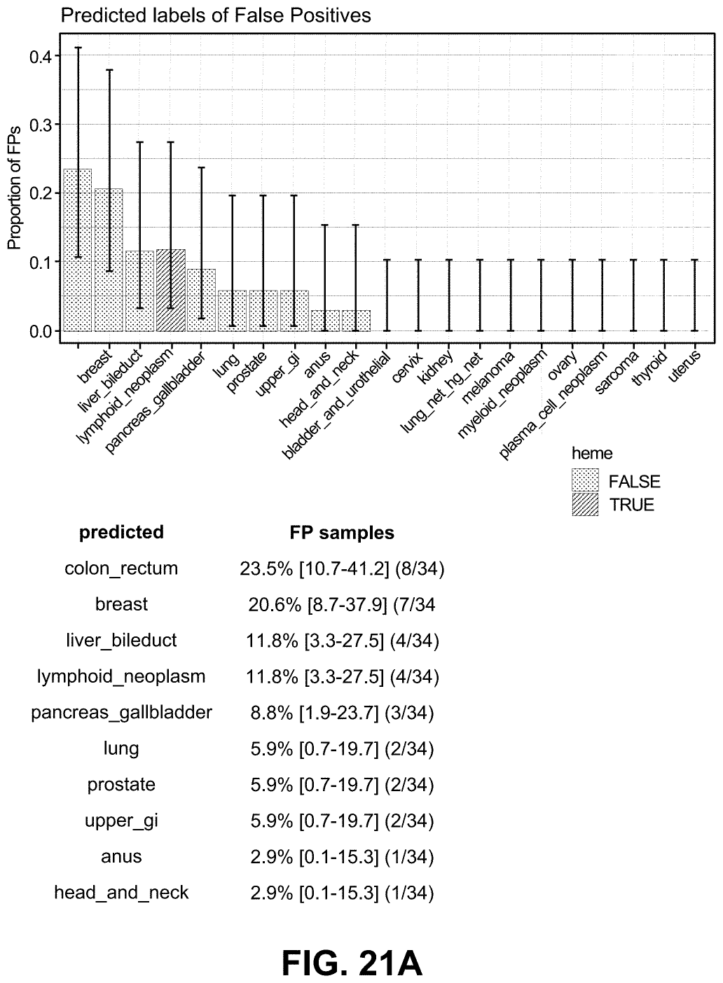

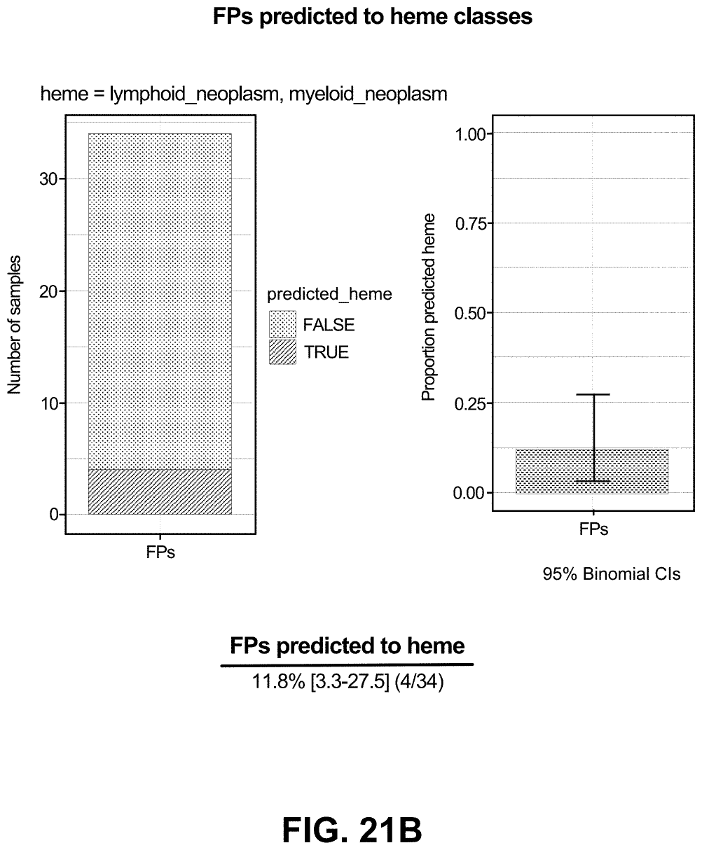

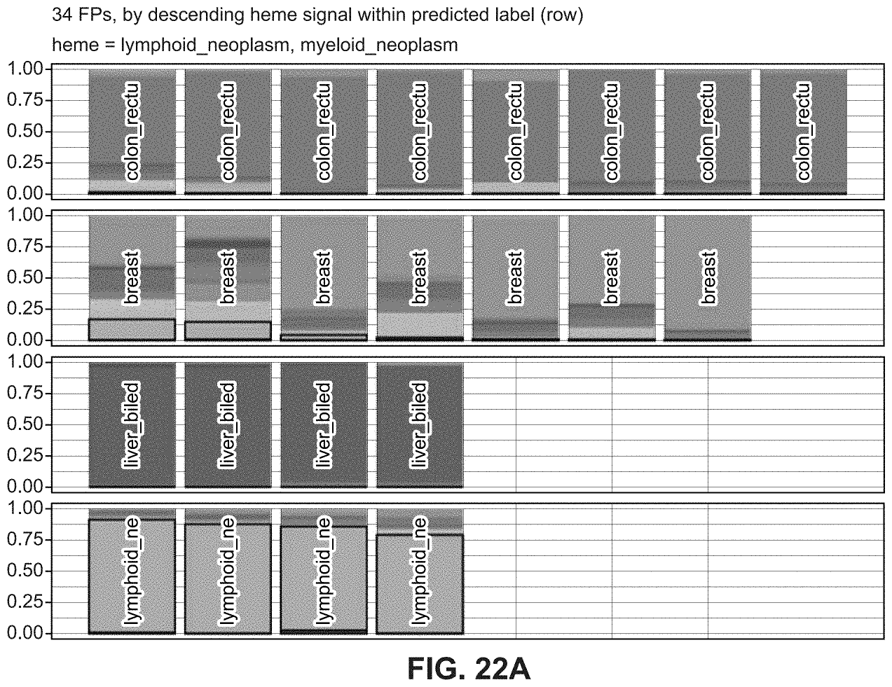

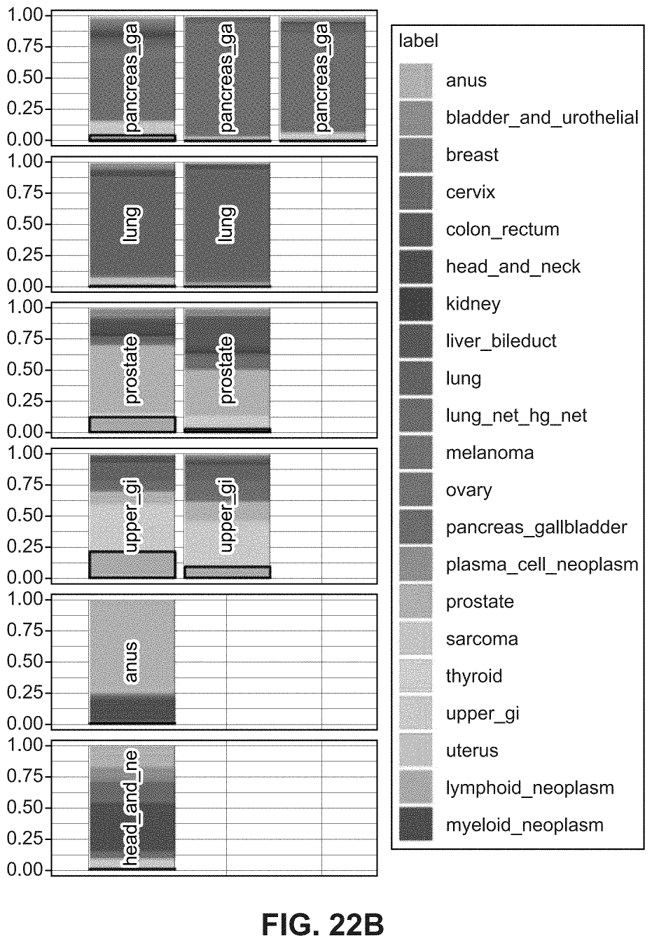

FIGS. 21A and 21B illustrate cancer signal localizations of false positives, according to some embodiments. FIGS. 22A and 22B illustrate cancer signal localizations of false positives based on cancer type, according to some embodiments. The results shown in FIGS. 21A-B indicate whether the false positive tissue of origin localizations are predicted to have hematological (blood) origins or solid (tumor) origins. The false positives are predominately predicted to solid localizations.

### V. CANCER APPLICATIONS

In some embodiments, the methods, analytic systems and/or classifier of the present disclosure can be used to detect the presence (or absence) of cancer, monitor cancer progression or recurrence, monitor therapeutic response or effectiveness, determine a presence or monitor minimum residual disease (MRD), or any combination thereof. In some embodiments, the analytic systems and/or classifier may be used to identify the tissue or origin for a cancer. For instance, the systems and/or classifiers may be used to identify a cancer as of any of the following cancer types: head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer. For example, as described herein, a classifier can be used to generate a likelihood or probability score (e.g., from 0% to 100%, or 0 to 100) that a sample feature vector is from a subject with cancer.

In some embodiments, the probability score is compared to a threshold probability to determine whether or not the subject has cancer. In other embodiments, the likelihood or probability score can be assessed at different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment. In some embodiments, a test report can be generated to provide a patient with their test results, including, for example, a probability score that the patient has a disease state (e.g., cancer), a type of disease (e.g., a type of cancer), and/or a disease tissue of origin (e.g., a cancer tissue of origin).

V.A. Early Detection of Cancer

In some embodiments, the methods and/or classifier of the present disclosure are used to detect the presence or absence of cancer in a subject suspected of having cancer. For example, a classifier (as described herein) can be used to determine a likelihood or probability score that a sample feature vector is from a subject that has cancer.

In one embodiment, a probability score of greater than or equal to 60 can indicated that the subject has cancer. In still other embodiments, a probability score greater than or equal to 65, greater than or equal to 70, greater than or equal to 75, greater than or equal to 80, greater than or equal to 85, greater than or equal to 90, or greater than or equal to 95, indicated that the subject has cancer. In other embodiments, a probability score can indicate the severity of disease. For example, a probability score of 80 may indicate a more severe form, or later stage, of cancer compared to a score below 80 (e.g., a score of 70). Similarly, an increase in the probability score over time (e.g., at a second, later time point) can indicate disease progression or a decrease in the probability score over time (e.g., at a second, later time point) can indicate successful treatment.

In another embodiment, a cancer log-odds ratio can be calculated for a test subject by taking the log of a ratio of a probability of being cancerous over a probability of being non-cancerous (i.e., one minus the probability of being cancerous), as described herein. In accordance with this embodiment, a cancer log-odds ratio greater than 1 can indicate that the subject has cancer. In still other embodiments, a cancer log-odds ratio greater than 1.2, greater than 1.3, greater than 1.4, greater than 1.5, greater than 1.7, greater than 2, greater than 2.5, greater than 3, greater than 3.5, or greater than 4, indicated that the subject has cancer. In other embodiments, a cancer log-odds ratio can indicate the severity of disease. For example, a cancer log-odds ratio greater than 2 may indicate a more severe form, or later stage, of cancer compared to a score below 2 (e.g., a score of 1). Similarly, an increase in the cancer log-odds ratio over time (e.g., at a second, later time point) can indicate disease progression or a decrease in the cancer log-odds ratio over time (e.g., at a second, later time point) can indicate successful treatment.

According to aspects of the disclosure, the methods and systems of the present disclosure can be trained to detect or classify multiple cancer indications. For example, the methods, systems and classifiers of the present disclosure can be used to detect the presence of one or more, two or more, three or more, five or more, or ten or more different types of cancer.

V.B. Cancer and Treatment Monitoring

In certain embodiments, the first time point is before a cancer treatment (e.g., before a resection surgery or a therapeutic intervention), and the second time point is after a cancer treatment (e.g., after a resection surgery or therapeutic intervention), and the method utilized to monitor the effectiveness of the treatment. For example, if the second likelihood or probability score decreases compared to the first likelihood or probability score, then the treatment is considered to have been successful. However, if the second likelihood or probability score increases compared to the first likelihood or probability score, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the disclosure to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

V.C. Treatment

In still another embodiment, information obtained from any method described herein (e.g., the likelihood or probability score) can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment (e.g., a resection surgery, radiation therapy, chemotherapy, and/or immunotherapy). In some embodiments, information such as a likelihood or probability score can be provided as a readout to a physician or subject.

A classifier (as described herein) can be used to determine a likelihood or probability score that a sample feature vector is from a subject that has cancer. In one embodiment, an appropriate treatment (e.g., resection surgery or therapeutic) is prescribed when the likelihood or probability exceeds a threshold. For example, in one embodiment, if the likelihood or probability score is greater than or equal to 60, one or more appropriate treatments are prescribed. In another embodiments, if the likelihood or probability score is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, a cancer log-odds ratio can indicate the effectiveness of a cancer treatment. For example, an increase in the cancer log-odds ratio over time (e.g., at a second, after treatment) can indicate that the treatment was not effective. Similarly, a decrease in the cancer log-odds ratio over time (e.g., at a second, after treatment) can indicate successful treatment. In another embodiment, if the cancer log-odds ratio is greater than 1, greater than 1.5, greater than 2, greater than 2.5, greater than 3, greater than 3.5, or greater than 4, one or more appropriate treatments are prescribed.

In some embodiments, the treatment is one or more cancer therapeutic agents selected from the group including a chemotherapy agent, a targeted cancer therapy agent, a differentiating therapy agent, a hormone therapy agent, and an immunotherapy agent. For example, the treatment can be one or more chemotherapy agents selected from the group including alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group including signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group including anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### VI. ADDITIONAL CONSIDERATIONS

The foregoing description of the embodiments of the disclosure has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the disclosure in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In some embodiments, a software module is implemented with a computer program product including a computer-readable non-transitory medium containing computer program code, which can be executed by a computer processor for performing any or all of the steps, operations, or processes described.

Embodiments can also relate to a product that is produced by a computing process described herein. Such a product can include information resulting from a computing process, where the information is stored on a non-transitory, tangible computer readable storage medium and can include any embodiment of a computer program product or other data combination described herein.

Finally, the language used in the specification has been principally selected for readability and instructional purposes, and it cannot have been selected to delineate or circumscribe the inventive subject matter. It is therefore intended that the scope of the invention be limited not by this detailed description, but rather by any claims that issue on an application based hereon. Accordingly, the disclosure of the embodiments herein is intended to be illustrative, but not limiting, of the scope of the invention, which is set forth in the following claims.

1. A method for cancer diagnosis comprising:
receiving a first plurality of cancer signals of a first sample of a first individual, wherein each one of the first plurality of cancer signals indicates a probability that the first sample is associated with a different disease state of a plurality of disease states;
determining a first cancer signal having a greatest probability among the first plurality of cancer signals;
responsive to determining that the first cancer signal satisfies a criterion, associating the first sample with a disease state corresponding to the first cancer signal;
providing, for presentation on a client device to determine a first diagnosis of the first individual, the disease state corresponding to the first cancer signal associated with the first sample;
receiving a second plurality of cancer signals of a second sample of a second individual, wherein each one of the second plurality of cancer signals indicates a probability that the second sample is associated with a different disease state of the plurality of disease states;
determining a second cancer signal having a greatest probability among the second plurality of cancer signals;
responsive to determining that the second cancer signal does not satisfy the criterion, associating the second sample with a subset of the plurality of disease states corresponding to a subset of the second plurality of cancer signals including at least the second cancer signal; and
providing, for presentation on the client device to determine a second diagnosis of the second individual, the subset of the plurality of disease states corresponding to the subset of the second plurality of cancer signals associated with the second sample.

2. The method of claim 1, further comprising:
determining a third cancer signal having a second greatest probability among the second plurality of cancer signals, wherein the subset of the second plurality of cancer signals further includes the third cancer signal.

3. The method of claim 1, wherein the criterion is a probability threshold, and wherein determining that the first cancer signal satisfies the criterion comprises:
determining that the greatest probability of the first cancer signal is greater than the probability threshold.

4. The method of claim 3, wherein the probability threshold is at least 90%.

5. The method of claim 1, further comprising:
determining the criterion based on accuracy of cancer signal probabilities and false positives.

6. The method of claim 1, further comprising:
determining the criterion based on residual risk of current cancer being associated with a sample.

7. The method of claim 1, further comprising:
determining a subset of n cancer signals of the first plurality of cancer signals having the n greatest probabilities among the first plurality of cancer signals; and
responsive to determining that at least a threshold number of the subset of the first plurality of cancer signals is associated with a category of disease states, associating the first sample with each disease state of the category of disease states.

8. The method of claim 7, wherein the category of disease states is human papillomavirus (HPV) cancer.

9. The method of claim 7, wherein the category of disease states includes stomach cancer and intestinal cancer.

10. The method of claim 1, wherein the plurality of disease states includes a non-cancer state.

11. The method of claim 1, wherein the plurality of disease states includes one or more types of cancer selected from the group including anus cancer, breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, leukemia, kidney cancer, liver cancer, bile duct cancer, plasma cell neoplasm cancer, upper gastrointestinal tract cancer, vulvar cancer, and lung neuroendocrine tumors and other high-grade neuroendocrine tumors.

12. The method of claim 1, further comprising:
providing, for presentation on the client device, a graphical comparison of each disease state corresponding to the subset of the plurality of disease states associated with the second sample.

13. The method of claim 12, wherein the graphical comparison is a bar plot based on the probabilities of the second plurality of cancer signals.

14-28. (canceled)

29. A system comprising a computer processor and a memory, the memory storing computer program instructions that when executed by the computer processor cause the processor to perform steps comprising the steps of:
receiving a first plurality of cancer signals of a first sample of a first individual, wherein each one of the first plurality of cancer signals indicates a probability that the first sample is associated with a different disease state of a plurality of disease states;
determining a first cancer signal having a greatest probability among the first plurality of cancer signals;
responsive to determining that the first cancer signal satisfies a criterion, associating the first sample with a disease state corresponding to the first cancer signal;
providing, for presentation on a client device to determine a first diagnosis of the first individual, the disease state corresponding to the first cancer signal associated with the first sample;
receiving a second plurality of cancer signals of a second sample of a second individual, wherein each one of the second plurality of cancer signals indicates a probability that the second sample is associated with a different disease state of the plurality of disease states;
determining a second cancer signal having a greatest probability among the second plurality of cancer signals;
responsive to determining that the second cancer signal does not satisfy the criterion, associating the second sample with a subset of the plurality of disease states corresponding to a subset of the second plurality of cancer signals including at least the second cancer signal; and
providing, for presentation on the client device to determine a second diagnosis of the second individual, the subset of the plurality of disease states corresponding to the subset of the second plurality of cancer signals associated with the second sample.

30. A non-transitory computer readable medium comprising instructions that, when executed by one or more processors, cause the one or more processors to perform steps comprising:
receiving a first plurality of cancer signals of a first sample of a first individual, wherein each one of the first plurality of cancer signals indicates a probability that the first sample is associated with a different disease state of a plurality of disease states;
determining a first cancer signal having a greatest probability among the first plurality of cancer signals;
responsive to determining that the first cancer signal satisfies a criterion, associating the first sample with a disease state corresponding to the first cancer signal;
providing, for presentation on a client device to determine a first diagnosis of the first individual, the disease state corresponding to the first cancer signal associated with the first sample;
receiving a second plurality of cancer signals of a second sample of a second individual, wherein each one of the second plurality of cancer signals indicates a probability that the second sample is associated with a different disease state of the plurality of disease states;
determining a second cancer signal having a greatest probability among the second plurality of cancer signals;
responsive to determining that the second cancer signal does not satisfy the criterion, associating the second sample with a subset of the plurality of disease states corresponding to a subset of the second plurality of cancer signals including at least the second cancer signal; and
providing, for presentation on the client device to determine a second diagnosis of the second individual, the subset of the plurality of disease states corresponding to the subset of the second plurality of cancer signals associated with the second sample.

31-34. (canceled)

35. The non-transitory computer readable medium of claim 30, comprising further instructions that when executed by the one or more processors, cause the one or more processors to perform steps comprising:
determining a third cancer signal having a second greatest probability among the second plurality of cancer signals, wherein the subset of the second plurality of cancer signals further includes the third cancer signal.

36. The non-transitory computer readable medium of claim 30, wherein the criterion is a probability threshold, and wherein determining that the first cancer signal satisfies the criterion comprises:
determining that the greatest probability of the first cancer signal is greater than the probability threshold.

37. The non-transitory computer readable medium of claim 30, comprising further instructions that when executed by the one or more processors, cause the one or more processors to perform steps comprising:
determining a subset of n cancer signals of the first plurality of cancer signals having the n greatest probabilities among the first plurality of cancer signals; and
responsive to determining that at least a threshold number of the subset of the first plurality of cancer signals is associated with a category of disease states, associating the first sample with each disease state of the category of disease states.

38. The non-transitory computer readable medium of claim 30, wherein the plurality of disease states includes a non-cancer state.

39. The non-transitory computer readable medium of claim 30, comprising further instructions that when executed by the one or more processors, cause the one or more processors to perform steps comprising:
providing, for presentation on the client device, a graphical comparison of each disease state corresponding to the subset of the plurality of disease states associated with the second sample.
